(19) United States
(12) Patent Application Publication
(10) Pub. No.: US 2013/0195797 A1
(43) Pub. Date: Aug. 1, 2013

(54) HIGH POTENCY FORMULATIONS OF VX-950

(71) Applicant: VERTEX PHARMACEUTICALS INCORPORATED, Cambridge, MA (US)

(72) Inventors: Eleni Dokou, Cambridge, MA (US); Geoffrey Glen BRODEUR, Somerville, MA (US); Michael TAUBER, Allston, MA (US); Tapan SANGHVI, Watertown, MA (US); Dragutin KNEZIC, Watertown, MA (US); Yolanda PESHA, Dedham, MA (US); Meghan M. JOHNSTON, Wakefiled, MA (US); Michael HURREY, Maynard, MA (US); Sneha Ghanshyam AREKAR, Brighton, MA (US); Rajesh PENUMATCHA, Woburn, MA (US)

(73) Assignee: VERTEX PHARMACEUTICALS INCORPORATED, Cambridge, MA (US)

(21) Appl. No.: 13/754,365

(22) Filed: Jan. 30, 2013

Related U.S. Application Data
(60) Provisional application No. 61/592,866, filed on Jan. 31, 2012.

(51) Int. Cl. A61K 9/16; A61K 31/381; A61K 38/21; A61K 31/7056; A61K 38/08; A61K 38/00

(52) U.S. Cl. CPC A61K 9/16; A61K 38/08; A61K 38/005; A61K 38/21; A61K 31/7056; A61K 31/381
USPC 424/85.4; 514/4.3

(57) ABSTRACT

High potency pharmaceutical compositions comprising VX-950, sodium lauryl sulfate and a polymer selected from the group consisting of hypromellose acetate succinate-M, hypromellose acetate succinate-L and hypromellose acetate succinate-H.

### CROSS-REFERENCE

The present application claims priority to U.S. Application No. 61/592,866, filed on Jan. 31, 2012, the contents of which are incorporated herein by reference in its entirety.

### FIELD OF THE INVENTION

The present invention relates to novel spray dried dispersions and pharmaceutical compositions of VX-950 for oral administration. This invention also relates to high potency pharmaceutical compositions comprising VX-950, sodium lauryl sulfate and a polymer selected from the group consisting of hypromellose acetate succinate-M, hypromellose acetate succinate-L and hypromellose acetate succinate-H.

### BACKGROUND OF THE INVENTION

Hepatitis C virus (HCV) is estimated to infect 170 million people worldwide. Nearly four million individuals may be infected in the United States alone.

VX-950 is a competitive, reversible peptidomimetic hepatitis C virus NS3/4A protease inhibitor with a steady state binding constant (ki*) of 3 nM (and with a Ki of 8 nM) [See International Publication No. 02/018369].

In clinical trials, VX-950 has shown antiviral activity and been shown to be an effective therapy against HCV, which is recognized as the causative agent for most cases of non-A, non-B hepatitis, with an estimated human sero-prevalence of 3% globally.

Orally administerable, a drug including VX-950 can be provided to the patient in various dosage forms, including formations such as capsules, caplets, tablets and other solid forms.

Incivek™ is the commercially available pharmaceutical composition of VX-950, also known as telaprevir. The current FDA-approved dosage is 750 mg, 3 times per day. Each dose of Incivek™ includes 2, 375 mg tablets.

Swallowing such solid forms is a problem for many people, including children and geriatric patients. For example, when the solid form of the drug is large, or the dosage requires multiple capsules, caplets, tablets or other solid forms, swallowing such drug form can be difficult. To facilitate a less burdensome administration of such solid forms of drugs for affected patients, higher drug load formulations have been conceived. The result is that fewer pills are required to accommodate the same dosage as commercially available forms of VX-950. Such a formulation increases the probability of patient compliance with the prescribed dosage regimen, and also enables combination therapy, either through co-formulation or co-administration with other active pharmaceutical ingredients (API's).

### SUMMARY OF THE INVENTION

In one embodiment, the invention provides a spray-dried dispersion comprising VX-950, sodium lauryl sulfate and a polymer selected from the group consisting of hypromellose acetate succinate-M, hypromellose acetate succinate-L and hypromellose acetate succinate-H.

In some embodiments, the spray-dried dispersion comprises from about 40% to about 80% wt/wt VX-950. In some embodiments, the spray-dried dispersion comprises about 60% wt/wt VX-950. In some embodiments, the spray-dried dispersion comprises about 70% wt/wt VX-950.

In some embodiments, the spray-dried dispersion comprises from about 20% to about 50% wt/wt hypromellose acetate succinate-M. In some embodiments, the spray-dried dispersion comprises about 29% wt/wt hypromellose acetate succinate-M. In some embodiments, the spray-dried dispersion comprises about 39% wt/wt hypromellose acetate succinate-M.

In some embodiments, the spray-dried dispersion comprises from about 20% to about 50% wt/wt hypromellose acetate succinate-L. In some embodiments, the spray-dried dispersion comprises about 29% wt/wt hypromellose acetate succinate-L. In some embodiments, the spray-dried dispersion comprises about 39% wt/wt hypromellose acetate succinate-L.

In some embodiments, the spray-dried dispersion comprises from about 20% to about 60% wt/wt hypromellose acetate succinate-H. In some embodiments, the spray-dried dispersion comprises about 29% wt/wt hypromellose acetate succinate-H. In some embodiments, the spray-dried dispersion comprises about 39% wt/wt hypromellose acetate succinate-H.

In some embodiments, the spray-dried dispersion comprises about 1% wt/wt sodium lauryl sulfate.

In some embodiments, the spray-dried dispersion comprises about 60% wt/wt VX-950, about 39% hypromellose acetate succinate-M and about 1% sodium lauryl sulfate.

In some embodiments, the spray-dried dispersion comprises about 70% wt/wt VX-950, about 29% hypromellose acetate succinate-M and about 1% sodium lauryl sulfate.

In one embodiment, the invention provides a spray-dried dispersion of VX-950 comprising VX-950, sodium lauryl sulfate and two or more polymers selected from the group consisting of hypromellose acetate succinate-H, hypromellose acetate succinate-M and hypromellose acetate succinate-L.

In one embodiment, the invention provides a pharmaceutical composition comprising a spray-dried dispersion of VX-950, the spray dried dispersion comprising VX-950, sodium lauryl sulfate and a polymer selected from the group consisting of hypromellose acetate succinate-H, hypromellose acetate succinate-M and hypromellose acetate succinate-L.

In some embodiments of the pharmaceutical composition, the spray-dried dispersion comprises from about 40% to about 80% wt/wt VX-950. In some embodiments, the spray-dried dispersion comprises about 60% wt/wt VX-950. In some embodiments, the spray-dried dispersion comprises about 70% wt/wt VX-950.

In some embodiments of the pharmaceutical composition, the spray-dried dispersion comprises from about 20% to about 50% wt/wt hypromellose acetate succinate-M. In some embodiments, the spray-dried dispersion comprises about 29% wt/wt hypromellose acetate succinate-M. In some embodiments, the spray-dried dispersion comprises about 39% wt/wt hypromellose acetate succinate-M.

In some embodiments of the pharmaceutical composition, the spray-dried dispersion comprises from about 20% to about 50% wt/wt hypromellose acetate succinate-L. In some embodiments, the spray-dried dispersion comprises about 29% wt/wt hypromellose acetate succinate-L. In some embodiments, the spray-dried dispersion comprises about 39% wt/wt hypromellose acetate succinate-L.

In some embodiments of the pharmaceutical composition, the spray-dried dispersion comprises from about 20% to about 60% wt/wt hypromellose acetate succinate-H. In some embodiments, the spray-dried dispersion comprises about 29% wt/wt hypromellose acetate succinate-H. In some embodiments, the spray-dried dispersion comprises about 39% wt/wt hypromellose acetate succinate-H.

In some embodiments of the pharmaceutical composition, the spray-dried dispersion comprises about 1% wt/wt sodium lauryl sulfate.

In some embodiments of the pharmaceutical composition, the spray-dried dispersion comprises about 60% wt/wt VX-950, about 39% hypromellose acetate succinate-M and about 1% sodium lauryl sulfate.

In some embodiments of the pharmaceutical composition, the spray-dried dispersion comprises about 70% wt/wt VX-950, about 29% hypromellose acetate succinate-M and about 1% sodium lauryl sulfate.

In one embodiment, the invention provides a pharmaceutical composition comprising a first spray-dried dispersion comprising VX-950, hypromellose acetate succinate-M and sodium lauryl sulfate and a second spray-dried dispersion comprising VX-950, hypromellose acetate succinate-H and sodium lauryl sulfate.

In one embodiment, the invention provides a pharmaceutical composition comprising a first spray-dried dispersion comprising VX-950, hypromellose acetate succinate-M and sodium lauryl sulfate and a second spray-dried dispersion comprising VX-950, hypromellose acetate succinate-L and sodium lauryl sulfate.

In one embodiment, the invention provides a pharmaceutical composition comprising a first spray-dried dispersion comprising VX-950, hypromellose acetate succinate-L and sodium lauryl sulfate and a second spray-dried dispersion comprising VX-950, hypromellose acetate succinate-H and sodium lauryl sulfate.

In some embodiments of the pharmaceutical composition, the first spray-dried dispersion comprises from about 40% to about 80% VX-950. In some embodiments, the first spray-dried dispersion comprises about 70% wt/wt VX-950. In some embodiments, the first spray-dried dispersion comprises about 60% wt/wt VX-950.

In some embodiments of the pharmaceutical composition, the second spray-dried dispersion comprises about 49.5% wt/wt VX-950.

In some embodiments of the pharmaceutical composition, the first spray-dried dispersion comprises from about 20% to about 50% hypromellose acetate succinate-M. In some embodiments, the first spray-dried dispersion comprises about 29% hypromellose acetate succinate-M. In some embodiments, the first spray-dried dispersion comprises about 39% hypromellose acetate succinate-M.

In some embodiments of the pharmaceutical composition, the first spray-dried dispersion comprises from about 50% to about 80% hypromellose acetate succinate-L.

In some embodiments of the pharmaceutical composition, the second spray-dried dispersion comprises from about 50% to about 80% hypromellose acetate succinate-L.

In some embodiments of the pharmaceutical composition, the first spray-dried dispersion comprises about 1% sodium lauryl sulfate.

In some embodiments of the pharmaceutical composition, the second spray-dried dispersion comprises about 1% sodium lauryl sulfate.

In some embodiments of the pharmaceutical composition, the first spray-dried dispersion comprises about 70% wt/wt VX-950, about 29% hypromellose acetate succinate-M and about 1% sodium lauryl sulfate.

In some embodiments of the pharmaceutical composition, the first spray-dried dispersion comprises about 60% wt/wt VX-950, about 39% hypromellose acetate succinate-M and about 1% sodium lauryl sulfate.

In some embodiments of the pharmaceutical composition, the second spray-dried dispersion comprises about 49.5% wt/wt VX-950, about 49.5% hypromellose acetate succinate-H and about 1% sodium lauryl sulfate.

In some embodiments, the pharmaceutical composition further comprises one or more of a diluent, a disintegrant, a flow agent and a lubricant.

In some embodiments, the pharmaceutical composition comprises a diluent. In some embodiments, the diluent is one or more of microcrystalline cellulose and anhydrous dibasic calcium phosphate.

In some embodiments, the pharmaceutical composition comprises a disintegrant. In some embodiments, the disintegrant is one or more of microcrystalline cellulose and croscarmellose sodium.

In some embodiments, the pharmaceutical composition comprises a flow agent. In some embodiments, the flow agent is colloidal silicon dioxide.

In some embodiments, the pharmaceutical composition comprises a lubricant. In some embodiments, the lubricant is sodium stearyl fumarate.

In one embodiment, the invention provides a process for preparing a solid dispersion of VX-950, the process comprising: a) forming a mixture comprising VX-950, a solvent and a polymer selected from the group consisting of hypromellose acetate succinate-M, hypromellose acetate succinate-L and hypromellose acetate succinate-H; and b) spray-drying the mixture to form a solid dispersion comprising VX-950.

In some embodiments of the process, the solid dispersion comprises from about 40% to about 80% wt/wt VX-950. In some embodiments, the solid dispersion comprises about 60% wt/wt VX-950. In some embodiments, the solid dispersion comprises about 70% wt/wt VX-950.

In some embodiments of the process, the solid dispersion comprises from about 20% to about 50% wt/wt hypromellose acetate succinate-M. In some embodiments, the solid dispersion comprises about 29% wt/wt hypromellose acetate succinate-M. In some embodiments, the solid dispersion comprises about 39% wt/wt hypromellose acetate succinate-M.

In some embodiments of the process, the solid dispersion comprises from about 20% to about 50% wt/wt hypromellose acetate succinate-L. In some embodiments, the solid dispersion comprises about 29% wt/wt hypromellose acetate succinate-L. In some embodiments, the solid dispersion comprises about 39% wt/wt hypromellose acetate succinate-L.

In some embodiments of the process, the solid dispersion comprises from about 20% to about 60% wt/wt hypromellose acetate succinate-H. In some embodiments, the solid dispersion comprises about 49.5% wt/wt hypromellose acetate succinate-H.

In some embodiments of the process, the solid dispersion comprises about 1% wt/wt sodium lauryl sulfate.

In some embodiments of the process, the solvent is one or more of methylene chloride, acetone and water.

In one embodiment, the invention provides a process for preparing a pharmaceutical composition comprising VX-950, the process comprising: a) forming a first mixture comprising VX-950, a solvent and a polymer selected from the group consisting of hypromellose acetate succinate-M, hypromellose acetate succinate-L and hypromellose acetate succinate-H; b) spray-drying the mixture to form a first solid dispersion comprising VX-950; c) forming a second mixture comprising VX-950, a solvent and a polymer selected from the group consisting of hypromellose acetate succinate-M, hypromellose acetate succinate-L and hypromellose acetate succinate-H; d) spray-drying the mixture to form a second solid dispersion comprising VX-950; and e) combining the first and second solid dispersions to form a pharmaceutical composition comprising VX-950.

In some embodiments of the process, the first or second solid dispersion comprises from about 50% to about 80% wt/wt VX-950. In some embodiments, the first or second solid dispersion comprises about 60% wt/wt VX-950 In some embodiments of the process, the first or second solid dispersion comprises about 70% wt/wt VX-950.

In some embodiments of the process, the first or second solid dispersion comprises from about 20% to about 50% wt/wt hypromellose acetate succinate-M. In some embodiments, the first or second solid dispersion comprises about 29% wt/wt hypromellose acetate succinate-M. In some embodiments, the first or second solid dispersion comprises about 39% wt/wt hypromellose acetate succinate-M.

In some embodiments of the process, the first or second solid dispersion comprises from about 20% to about 50% wt/wt hypromellose acetate succinate-L. In some embodiments, the first or second solid dispersion comprises about 29% wt/wt hypromellose acetate succinate-L. In some embodiments, the first or second solid dispersion comprises about 39% wt/wt hypromellose acetate succinate-L.

In some embodiments of the process, the first or second solid dispersion comprises from about 20% to about 60% wt/wt hypromellose acetate succinate-H. In some embodiments, the first or second solid dispersion comprises about 49.5% wt/wt hypromellose acetate succinate-H.

In some embodiments of the process, the first or second solid dispersion comprises about 1% wt/wt sodium lauryl sulfate.

In one embodiment, the invention provides a method of treating a patient infected with the hepatitis C virus, the method comprising administering the pharmaceutical composition of any one of the preceding embodiments.

In some embodiments, the method further comprises administering one or more additional antiviral agents. In some embodiments, the method further comprises administering pegylated interferon. In some embodiments, the method further comprises administering ribavirin. In some embodiments, the method further comprises administering VX-222.

In some embodiments, the pharmaceutical composition comprises about 250 mg to about 2250 mg VX-950 per administration.

In some embodiments, the pharmaceutical formulation is administered: a) in an amount of 250 mg VX-950; b) in an amount of 300 mg VX-950; c) in an amount of 400 mg VX-950; d) in an amount of 450 mg VX-950; e) in an amount of 500 mg VX-950; f) in an amount of 600 mg VX-950; g) in an amount of 650 mg VX-950; h) in an amount of 750 mg VX-950; i) in an amount of 850 mg VX-950; j) in an amount of 1000 mg VX-950; or k) in an amount of 1250 mg VX-950; or 1) in an amount of 2250 mg VX-950.

In some embodiments, the pharmaceutical formulation is administered once per day, twice per day or three times per day.

In some embodiments, the spray-dried dispersion provides an average plasma concentration (C_{avg}) of VX-950 of at least about 750 ng/mL after administration to a human. In some embodiments, the average plasma concentration (C_{avg}) of VX-950 is about 750 ng/mL to about 1250 ng/mL after administration to a human. In some embodiments, the pharmaceutical composition provides an average plasma concentration (C_{avg}) of VX-950 of at least about 750 ng/mL after administration to a human.

In some embodiments, the pharmaceutical composition provides an average plasma concentration (C_{avg}) of VX-950 of at least about 750 ng/mL to about 1250 ng/mL after administration to a human.

In some embodiments, the spray-dried dispersion provides at least about a 2 log₁₀ decrease of hepatitis C virus RNA in the plasma when administered to a human. In some embodiments, the spray-dried dispersion provides at least about a 4 log₁₀ decrease of hepatitis C virus RNA in the plasma when administered to a human.

In some embodiments, the pharmaceutical composition provides at least about a 2 log₁₀ decrease of hepatitis C virus RNA in the plasma when administered to a human. In some embodiments, the pharmaceutical composition provides at least about a 4 log₁₀ decrease of hepatitis C virus RNA in the plasma when administered to a human.

In some embodiments, the pharmaceutical composition is substantially bioequivalent to Incivek™.

In some embodiments, the 90% confidence interval of the relative mean Cₘₐₓ of the pharmaceutical composition to Incivek™ is substantially within 80% to 125.

In some embodiments, the 90% confidence interval of the relative mean AUC₍₀₋ₜ₎ of the pharmaceutical composition to Incivek™ is substantially within 80% to 125%.

In some embodiments, the 90% confidence interval of the relative mean AUC_{(0-∞)} of the pharmaceutical composition to Incivek™ is substantially within 80% to 125%.

In some embodiments, the plurality of polymers decreases the amount of crystallization or rate of crystallization of the VX-950 by at least about 10% as compared to a spray-dried dispersion without being in the presence of the plurality of polymers.

In some embodiments, the plurality of polymers decreases the amount of crystallization or rate of crystallization of the VX-950 by at least about 10% as compared to a pharmaceutical composition without the presence of the plurality of polymers.

In some embodiments, the presence of hypromellose acetate succinate-M decreases the amount of crystallization or rate of crystallization of the VX-950 by at least about 10% as compared to a pharmaceutical composition without the presence of hypromellose acetate succinate-M.

In some embodiments, the presence of hypromellose acetate succinate-L decreases the amount of crystallization or rate of crystallization of the VX-950 by at least about 10% as compared to a pharmaceutical composition without the presence of hypromellose acetate succinate-L.

In some embodiments, presence of hypromellose acetate succinate-H decreases the amount of crystallization or rate of crystallization of the VX-950 by at least about 10% as compared to a pharmaceutical composition without the presence of hypromellose acetate succinate-H.

In some embodiments, the pharmaceutical composition further comprises an additional active pharmaceutical ingredient. In some embodiments, the additional active pharmaceutical ingredient is VX-222.

All of the documents cited herein, are incorporated herein by reference in their entireties.

### DESCRIPTION OF THE FIGURES

FIG. 1: Manufacturing Process Flow Diagram for Telaprevir Spray-dried Dispersion.

FIG. 2: Manufacturing Process Flow Diagram for Telaprevir 60% M tablet, 562.5 mg and Telaprevir 70% M tablet, 562.5 mg.

FIG. 3: Manufacturing Process Flow Diagram for Telaprevir 49.5% H+70% M tablet, 562.5 mg.

FIG. 4: Manufacturing Process Flow Diagram for Telaprevir 60% M Spray Dried Dispersion.

FIG. 5: Manufacturing Process Flow Diagram for Telaprevir 60% M Spray Dried Dispersion.

FIG. 6: Manufacturing Process Flow Diagram for Telaprevir 60% M tablet, 562.5 mg.

FIG. 7: Comparison of Kinetic Solubility of VX-950 tablets containing 60% SDDs with different grades of HPMCAS polymer (H, M and L) in FESSIF D<01> pH 5.0 and pH 6.8.

FIG. 8: Comparison of Kinetic Solubility of VX-950 tablets containing 49.5% H and 70% M SDD blend verses 60% M in FESSIF D<01> pH 5.0 and pH 6.8.

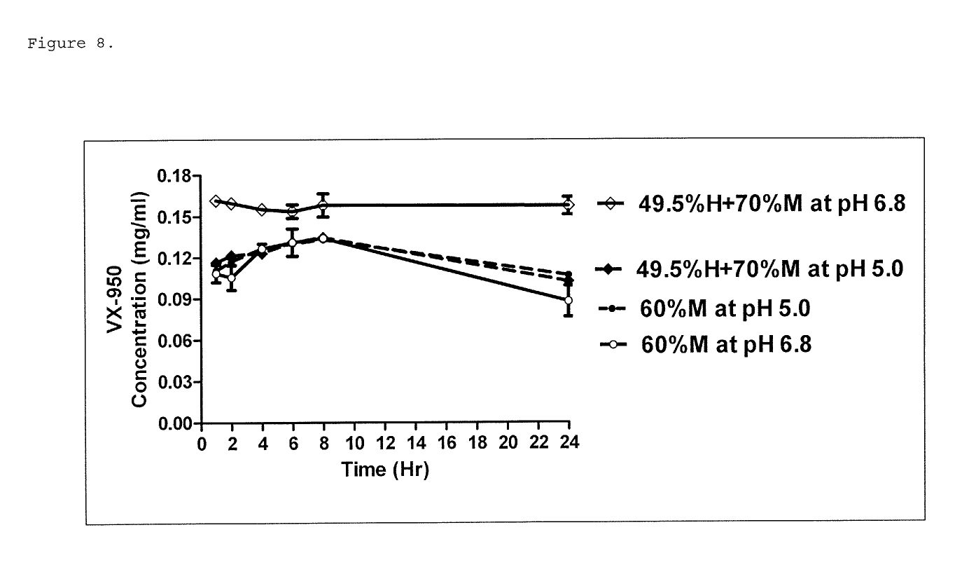

FIG. 9: Comparison of Kinetic Solubility of VX-950 tablets containing 70% L, 70% M and 70% H SDD in FESSIF D<01> pH 5.0 and pH 6.8.

### DESCRIPTION OF THE INVENTION

The pharmaceutical formulations of the present invention can be used as a delivery system for the administration of one or more APIs. Any suitable API can be used in accordance with the present invention.

VX-950 is described in PCT Publication Numbers WO 02/018369, WO 2006/050250 and WO/2008/144072, with reference to the following structural formula, or a pharmaceutically acceptable salt thereof:

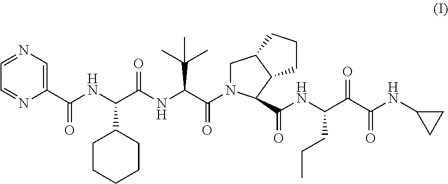 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

Other descriptions of VX-950 can be found in PCT Publication Numbers WO 07/098,270 and WO 08/106,151.

As used herein, the term “VX-950,” refers to the compound of Formula (I), or a pharmaceutically acceptable salt thereof. Further, the term “VX-950” can also include a processed form of VX-950. For example, a VX-950 spray-dried dispersion that includes VX-950 and a polymer(s) is encompassed within the term. A spray-dried dispersion of VX-950 is described in WO 05/123076, WO 07/109,604, WO 07/109,605 and WO 08/080,167.

In one aspect of the invention, VX-950 is in the form of a spray dried dispersion. The term “spray dried” or “spray drying” in the present specification means the state of the drug alone or the drug together with a pharmaceutically acceptable carrier dissolved in a solvent that is pharmaceutically acceptable, or suspended with the drug or part or all of the carrier dispersed in a solvent and this solution or suspension being sprayed and dried.

Spray drying of the pharmaceutical compositions may be undertaken utilizing either rotary, pneumatic or pressure atomisers located in either a co-current, counter-current or mixed-flow spray dryer or variations thereof.

The amount of VX-950 in the formulation of the present invention can be expressed in terms of a weight percentage. For example, the active ingredient in the formulation of the present invention can constitute from greater than 0% to about 80% by weight based on the total weight of the formulation, or from greater than 0% to about 60% by weight based on the total weight of the formulation. The amount of VX-950 in the formulation of the present invention also can be expressed in terms of total mass of the formulation. For example, the formulation of the present invention can include VX-950 in an amount of from about 1 μg to about 2 g per tablet, or from about 0.01 mg and about 1000 mg per tablet. In another example, the formulation of the present invention can include one or more active ingredients in amounts of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. In some embodiments, the formulation of the present invention can include one or more active ingredients in amounts of about 100 mg, about 250 mg. Or for example, the formulation of the present invention can include one or more active ingredients in amounts that range, e.g., from about 0.1 mg to about 0.5 mg, from about 1 mg to about 20 mg (e.g., 2 mg, 8 mg, 15 mg), from about 50 mg to about 100 mg (e.g., 80 mg), from about 100 mg to about 500 mg (e.g., 100 mg, 200 mg, 250 mg, 300 mg), from about 100 to 200 mg, from about 100 mg to 150 mg, from about 100 mg to about 125 mg, from about 200 mg to about 300 mg, from 200 mg to about 250 mg, from 225 mg to 250 mg, from about 225 mg to about 250 mg, from about 240 mg to about 250 mg, or from about 500 mg to about 1000 mg.

In some embodiments, the active ingredient constitutes from about 40% to about 80% of the mass of the spray-dried dispersion. In some embodiments, the active ingredient constitutes about 49.5%, about 60% or about 70% of the mass of the spray-dried dispersion. It is understood that about 40% encompasses a range from 38-42%; about 49.5% encompasses a range from 47.5-51.5%; about 60% encompasses a range from 58-620; about 70% encompasses a range from 68% to 72%; and about 80% encompasses a range from 78% to 82%.

“HPMCAS,” “hydroxypropyl methylcellulose acetate succinate” or “hypromellose acetate succinate” refers to a substituted cellulose derivative bearing succinoyl and acetyl groups. HPMCAS is a hydrophilic polymer used here in a pharmaceutical dosage form of the present invention. HPMCAS has unique dissolution characteristics that can be controlled by changing the ratio of succinoyl and acetyl groups, which affects the dissolution of the dosage forms and the subsequent drug release, as does the formulation amount of the agent. The succinoyl group content of HPMCAS determines the pH-solubility profile, and a reduction of the succinoyl group content results in an increase in the pH at which HPMCAS starts to dissolve. Of the three commercially available grades of HPMCAS, hypromellose acetate succinate-L (L), hypromellose acetate succinate-M (M) and hypromellose acetate succinate-H (H) (Table 1), the proportions of succinoyl substitution to acetyl substitution (SA ratio) is highest in L, which is soluble at lower pH, whereas H, having a low SA ratio, dissolves at higher pH. With decreasing succinoyl group content the pH at which each HPMCAS polymer starts to dissolve increases. The grade L and M, respectively, dissolution occurs at pHs above 5.5 and 6.0, respectively. The majority of commercial H grade of HPMCAS solubilizes above a pH of 6.5.

TABLE 1
Solubilization patterns of HPMCAS used for solid
dispersions
Content of substitutions %

| | | | | Hydroxy- | |
|---|---|---|---|---|---|
| Type | Succinoyl | Acetyl | Methoxyl | propoxyl | Soluble at pH of |
| L | 14.8 | 7.3 | 7.1 | 22.7 | 5.5 and higher |
| M | 11.0 | 9.3 | 7.4 | 23.0 | 6.0 and higher |
| H | 7.8 | 11.1 | 7.4 | 23.5 | 6.5 and higher |

In first embodiment of the dispersion, a dispersion of the drug is prepared in accordance with the methods described herein using one or more commercially available grades of HPMCAS. In a second embodiment of the dispersion, the dispersion can be prepared by mixing two or more dispersions of the first embodiment. It should be noted that the SA ratio may or may not be the same for this second embodiment of the dispersion compared to that of the first embodiment, but, because the two embodiments were processed differently (that is, for example, in the first embodiment, H and M grades of HPMCAS were co-mixed and dispersed together simultaneously in a same batch instead of mixing a dispersion of H grade of HPMCAS with that of M, as in the second embodiment), the dispersion of the second embodiment, when compared to the first, may have a different dissolution profile of the drug in a media of use.

It is understood that HPMCAS-M, L and H encompass MG, MF, LG, LF, HG and HF, respectively, as well as other commercially available variations of the M, L and H grades.

In some embodiments, HPMCAS-M, L or H constitutes from about 20% to about 60% of the mass of the spray-dried dispersion. In some embodiments, the HPMCAS constitutes about 49.5%, about 39% or about 29% of the mass of the spray-dried dispersion. It is understood that about 20% encompasses a range from 18-22%; about 49.5% encompasses a range from 47.5-51.5%; about 39% encompasses a range from 37-41%; about 29% encompasses a range from 27% to 31%; and about 60% encompasses a range from 58% to 62%.

In some embodiments of the present invention, the formulation of VX-950 is a pediatric formulation. The dosage and frequency of administration will depend on the age, sex and condition of the pediatric patient, concurrent administration of other drugs, contraindications and other parameters to be taken into account by the clinician.

The pharmaceutical formulations of this invention may be orally administered in any orally acceptable dosage form including, but not limited to, capsules, sprinkles, tablets, aqueous suspensions or solutions. In one embodiment of the present invention, the pharmaceutical formulation is in form of a tablet. Furthermore, in one embodiment, the tablet is swallowed in a solid form. In another embodiment, the tablet form can be a chewable, orally disintegrating and/or rapidly disintegrating form.

The term “tablet” refers to a pharmacological composition in the form of a small, essentially solid pellet of any shape. Tablet shapes can be cylindrical, spherical, rectangular, capsular or irregular. The term “tablet composition” refers to the substances included in a tablet. A “tablet composition constituent” or “tablet constituent” refers to a compound or substance which is included in a tablet composition. These can include, but are not limited to, the active ingredient and one or more excipients in addition.

The amounts of VX-950 according to this invention are administered in a single dosage form or in more than one dosage form. If in separate dosage forms, each dosage form is administered about simultaneously. For the avoidance of doubt, for dosing regimens calling for dosing more than once a day, one or more tablet or dose may be given at each time per day (e.g., 1 tablet, twice per day, 2 tablets, twice per day or 3 tablets, twice per day).

Methods of forming the tablets of the invention wherein all tablet constituents are combined simultaneously or wherein any combination of tablet constituents are combined separate from the other constituents are within the scope of the invention.

VX-950 and excipient(s) mixture can be prepared by, for instance, conventional mixing, compacting, granulating, compression, or coating. Procedures which may be used are known in the art, e.g., those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed. (Mack Publ., Co., 1970) or later editions. Examples of such techniques are as follows:

(1) Blending of VX-950 with the appropriate excipients using different blending equipment, such low shear blenders and high shear blenders;

(2) Direct compression of the blends, using appropriate punches and dies; the punches and dies are fitted to a suitable compaction machine, such as rotary tableting press or a single station compaction machine;

(3) The formulation blend can be granulated if necessary, using appropriate granulation methods such as dry granulation (slugging or roller compaction), high shear wet granulation, fluid bed granulation, extrusion-spheronization etc;

(4) Granulation followed by compression; and

(5) Coating of the tablets, if necessary, produced using appropriate coating equipment (e.g., coating pans) and appropriate coating solutions/suspensions to be applied on the tablets.

In one aspect of the present invention, the formulations of the present invention find their greatest utility when administered to a subject who is in the fed or fasted state, preferably in the fed state.

The tablets may be produced by way of a conventional method or combinations of conventional methods such as roller compaction and compression method. For example, a tableting process is essential for production methods of tablets, and also the other processes such as of mixing, drying, and coating may be combined as required. The tableting process may be, for example, a direct compression method where VX-950 and pharmaceutically acceptable excipients disclosed herein are mixed and then the mixture is compressed into tablets by use of tableting machines.

In one embodiment of the invention, the tablet has a hardness in the range of about 3 to 30 kp (kilopond). The tablet of this embodiment may or may not comprise an outer coating as described below.

Once tablet compositions are prepared, they may be formed into various shapes. In some embodiments, the tablet compositions are pressed into a shape. This process may comprise placing the tablet composition into a form and applying pressure to the composition so as to cause the composition to assume the shape of the surface of the form with which the composition is in contact. In some embodiments, the tablet has a hardness in the range of about 10 to 20 kp. In some embodiments, the tablet has a hardness in the range of about 12 to 17 kp.

Yet in one embodiment of the present invention, the formulation includes tablet compositions that may be coated.

The present invention can also provide a formulation that diminishes the bitter taste, mouth irritation, and dry mouthfeel when a patient is administered with VX-950.

The present invention is suitable for rendering VX-950 that are bitter tasting and/or throat catching. Taste improving compositions would diminish any off-flavors in the taste of VX-950, and to also improve the taste of any other off-flavor components included in the formulation if desired.

The term “taste improving” referred herein can be defined as a perceived reduction of an undesirable taste that would otherwise be there to making it possible to delay or diminish the occurrence of an unpleasant taste specific to a product during its oral, buccal or nasal administration.

In one embodiment, the taste improving composition can include one or more components. In some embodiments, the taste improving composition can include one or more sweeteners. In some embodiments, the taste improving composition can include one or more flavoring agents. In some embodiments, the taste improving composition can include a combination of a sweetener and a flavoring agent.

In some embodiments, one or more sweeteners include, but are not limited to, monosaccharides, disaccharides and polysaccharides. Examples of suitable sweeteners include both natural and artificial sweeteners. Examples can include, but are not limited to, glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartane, neotame, alitame, neohesperidin dihydrochalcone, sodium cyclamate, thaumatin, acesulfame potassium, saccharin, and saccharin sodium.

The flavoring agent used is of the type and amount desired to enhance the palatability of the particular liquid pharmaceutical composition to the intended consumer. The flavoring agent used for a solid formulation is similar.

Suitable flavoring agents can include, for example, flavors, which are known to those of skill in the art, such as, for example, natural flavors, artificial flavors, and combinations thereof. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavoring agents may be used in liquid or solid form and, as indicated above, may be used individually or in admixture. Other flavoring agents can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof. In one embodiment, the flavoring agent of the present invention is ethyl vanillin, natural & artificial orange flavor or both. A formulation of the present invention can include from about 0 wt. % to about 5 wt. % of the flavor agent. In some embodiments, a formulation of the present invention can include from about 1 wt. % to about 3 wt. % of the flavor agent. In some embodiments, a formulation of the present invention can include from about 2 wt. % to about 3 wt. % of the flavor agent.

The term “bioequivalent” refers to the United States Food and Drug Administration (FDA) guidelines for pharmaceutical formulations. It would be understood by a person of ordinary skill in the art that two products are bioequivalent if the 90% confidence interval (CI) of the relative mean Cₘₐₓ, AUC₍₀₋ₜ₎ and AUC_{(0-∞)} of the test formulation to the reference formulation is within 80.00% to 125.00%.

The term “filler component” refers to one or more substances that act to dilute the API to the desired dosage and/or that act as a carrier for the API. An “excipient” can also refer to a non-toxic pharmaceutically acceptable substance added to a pharmacological composition to facilitate the processing, administration and pharmaceutics properties of a compound. Excipients that are pharmaceutically acceptable and are used as additives can also be added to the pharmaceutical formulations of the present invention. Examples of these excipients are a filler/diluent (extender)/binder, disintegrant, sweetener, flavoring agent, lubricant, glidant, surfactant, coloration agent or a combination thereof. One or a combination of 2 or more of these excipients can be used. Other excipients include e.g. coloring agents, pH-adjusting agents, buffering agents, preservatives, anti-oxidants, wetting agents, humidity-adjusting agents, surface-active agents, suspending agents, absorption enhancing agents, foaming agents, agents for modified release and mixtures thereof. Generally, excipients forth may be used for customary purposes and in typical amounts without adversely affecting the properties of the compositions. These excipients may be utilized in order to formulate the composition into tablets, capsules, and other solid forms. In some embodiments, the filler component comprises at least one of a substance that improves the mechanical strength and/or compressibility of the pharmaceutical compositions of the invention.

Examples of the filler can include, but are not limited to, mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate. In one embodiment, the filler is dibasic calcium phosphate, microcrystalline cellulose, or a combination thereof.

In some embodiments, the filler is present in an amount of about at least 1, 2, 3, 4, 5, 6, 7, 8, 9, 10, 11, 12, 13, 14, 15, 20, 25, 30, 35, 40, 45 or 50% of the total weight of the formulation.

In certain embodiments, the pharmaceutical compositions of the present invention comprise a first filler and a second filler. In some embodiments of the pharmaceutical formulations, each of the first and second filler component independently comprises from about 0.01% to about 30% by weight of the pharmaceutical formulation. In some embodiments of the pharmaceutical formulations, each of the first and second filler component independently comprises from 5% to about 25% by weight of the pharmaceutical formulation. In some embodiments of the pharmaceutical formulations, each of the first and second filler component independently comprises from about 10% to about 20% by weight of the pharmaceutical formulation. In some embodiments of the pharmaceutical formulations, each of the first and second filler component independently comprises from about 15% to about 20% by weight of the pharmaceutical formulation. In some embodiments of the pharmaceutical formulations, each of the first and second filler component independently comprises from about 1% to about 20% by weight of the pharmaceutical formulation. In some embodiments of the pharmaceutical formulations, each of the first and second filler component independently comprises from about 1% to about 10% by weight of the pharmaceutical formulation. In some embodiments of the pharmaceutical formulations, each of the first and second filler component independently comprises from about 1% to about 5% by weight of the pharmaceutical formulation.

“Disintegrants” are substances that are added to a tablet to facilitate its breakup or disintegration after administration. Examples of the disintegrants may include, but are not limited to, croscarmellose sodium (e.g., AcDiSol), sodium alginate, calcium alginate, alginic acid, starch, pregelatinized starch, sodium starch glycolate, crospovidone, carboxymethylcellulose calcium, cellulose and its derivatives, carboxymethylcellulose sodium, microcrystalline cellulose, soy polysaccharide, guar gum, an ion exchange resin, an effervescent system based on food acids and an alkaline carbonate component, and sodium bicarbonate. In some embodiments of the pharmaceutical formulations, the disintegrant component is croscarmellose sodium.

In some embodiments of the pharmaceutical formulations, the disintegrant component comprises an amount of about at least 1, 2, 3, 4, 5, 6, 7, 8, 9, 10, 15, 20, 25, 30, 35 or 40% of the total weight of the formulation. In some embodiments of the pharmaceutical formulations, the disintegrant component comprises from about 0.01% to about 30% by weight of the pharmaceutical formulation. In some embodiments, the disintegrant component comprises from about 0.01% to about 20% by weight of the pharmaceutical formulation. In some embodiments, the disintegrant component comprises from about 0.5% to about 20% by weight of the pharmaceutical formulation. In some embodiments, the disintegrant component comprises from about 0.1% to about 20% by weight of the pharmaceutical formulation. In some embodiments, the disintegrant component comprises from about 0.5% to about 15% by weight of the pharmaceutical formulation. In some embodiments, the disintegrant component comprises from 0.5% to about 10% by weight of the pharmaceutical formulation. In some embodiments, the disintegrant component comprises from about 0.5% to about 5% by weight of the pharmaceutical formulation. In some embodiments, the disintegrant component comprises from about 3% to about 5% by weight of the pharmaceutical formulation. In some embodiments, the disintegrant component comprises from about 4% to about 5% by weight of the pharmaceutical formulation. In some embodiments, the disintegrant component comprises about 4.25% by weight of the pharmaceutical formulation. In some embodiments, the disintegrant component comprises from about 1% to about 4% by weight of the pharmaceutical formulation. In some embodiments, the disintegrant component comprises from about 1% to about 3% by weight of the pharmaceutical formulation. In some embodiments, the disintegrant component comprises from about 2% to about 3% by weight of the pharmaceutical formulation. In some embodiments, the disintegrant component comprises about 3% by weight of the pharmaceutical formulation. In some embodiments, the disintegrant component comprises about 3% by weight of the pharmaceutical formulation.

A “glidant” or “flow agent” is a substance to promote powder flow by reducing interparticle friction and cohesion. In certain embodiments, the one or more excipients can include one or more glidants. Examples of the glidants may include, but are not limited to, talc, colloidal silica (e.g., Cabosil M-5), magnesium oxide, magnesium silicate, leucine and starch. In one embodiment, the one or more glidants is colloidal silica. In one embodiment, the one or more glidants comprises about up to 3% by weight of the pharmaceutical formulation. In another embodiment, the one or more glidants comprises about up to 1% by weight of the pharmaceutical formulation. In another embodiment, the one or more glidants comprises from about 0.5% by weight to about 1% by weight of the pharmaceutical formulation. In another embodiment, the one or more glidants comprises about 0.85% by weight of the pharmaceutical formulation. In another embodiment, the one or more glidants comprises about up to 0.5% by weight of the pharmaceutical formulation.

In certain embodiments, the one or more excipients can include one or more lubricants. Suitable lubricants possess anti-sticking or anti-tacking properties. Examples of the lubricants may include, but are not limited to, talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, or a combination thereof. In certain embodiment of the pharmaceutical formulations, the one or more lubricant is sodium stearyl fumarate.

In some embodiments of the pharmaceutical formulations, the one or more lubricant comprises an amount of about at least 1, 2, 3, 4, 5, 6, 7, 8, 9, 10, 15, 20, 25, 30, 35 or 40% of the total weight of the formulation. In some embodiment, the one or more lubricant comprises from about 0.01% to about 30% by weight of the pharmaceutical formulation. In some embodiments, the one or more lubricant comprises from about 0.01% to about 20% by weight of the pharmaceutical formulation. In some embodiments, the one or more lubricant comprises from about 0.1% to about 20% by weight of the pharmaceutical formulation. In some embodiments, the one or more lubricant comprises from about 0.5% to about 5% by weight of the pharmaceutical formulation. In some embodiments, the one or more lubricant comprises from about 1% to about 5% by weight of the pharmaceutical formulation. In some embodiments, the one or more lubricant comprises from about 0.5% to about 4% by weight of the pharmaceutical formulation. In some embodiments, the one or more lubricant comprises from about 1% to about 3% by weight of the pharmaceutical formulation. In some embodiments, the one or more lubricant comprises about 3% by weight of the pharmaceutical formulation.

Examples of colorants are food coloring, such as yellow food dye No. 5, red food dye No. 2, blue food dye No. 2, etc.; food lake coloring; iron oxides (e.g. iron oxide red), etc.

In some embodiments, the one or more colorant comprises an amount of about at least 0.1, 0.5, 1, 2, 3, 4, 5, 6, 7, 8, 9, 10, 15 or 20% of the total weight of the composition. In one embodiment, the one or more colorants comprises from about 1% to about 5% by weight of the pharmaceutical formulation. In some embodiments, the one or more colorants comprises from about 0.5% to about 4% by weight of the pharmaceutical formulation. In some embodiments, the one or more colorants comprises from about 1% to about 3% by weight of the pharmaceutical formulation. In some embodiments, the one or more colorants comprises from about 0.1% by weight to about 3.0% by weight of the pharmaceutical formulation. In one embodiment, the formulation of the present invention includes red and yellow iron oxides comprising about 0.5% of the total weight of the composition.

Furthermore, an excipient disclosed herein can have more than one function. For example, mannitol can function as a sweetener as a component of the taste improving composition and/or as a filler.

Each dosage form may be individually housed, as in a sheet of a metal foil-plastic laminate with each dosage form isolated from the others in individual cells or bubbles, or the dosage forms may be housed in a single container, as in a plastic bottle. In some embodiments, the VX-950 is packaged in foil pouches with a polypropylene heat seal layer. In some embodiments, the VX-950 is packaged in high density polyethylene (HDPE) bottles.

In certain embodiments, a method according to this invention involves the treatment of a patient infected with genotype 1 Hepatitis C virus. In some embodiments, the patient is less than 18 years of age. In some embodiments, the patient is from 3 to 17 years of age. In some embodiments, the patient is from 18 to 50 years of age. In some embodiments, the patient is over 50 years of age.

In some embodiments, the patient is a treatment naïve patient. In other embodiments, the patient is a pegylated-interferon/ribavirin non-responder. As used herein “treatment naïve” refers to a patient who has not received any prior treatment for hepatitis C. As used herein “P/R non-responsive” includes patients who do not achieve or maintain a sustained virologic response (SVR) (undetectable HCV RNA 24 weeks after the completion of treatment) to the standard peg-IFN with RBV treatment, and patients who have had a lack of response. Lack of response is defined as a <2-log₁₀ decline from baseline in HCV RNA, as a failure to achieve undetectable levels of HCV virus, or as a relapse following discontinuation of treatment. As defined above, undetectable HCV RNA means that the HCV RNA is present in less than 10 IU/mL as determined by assays currently commercially available, for example, as determined by the Roche COBAS TaqMan™ HCV/HPS assay.

Any suitable dosage level of VX-950 can be employed in the formulations of the present invention. The dose to be administered to an animal, particularly a human, in accordance with the present invention should be sufficient to affect a therapeutic response in the animal over a reasonable time frame. One skilled in the art will recognize that the amount of active ingredient will vary depending upon a variety of factors including, for example, the activity of the specific compound employed; the age, body weight, general health, sex, and diet of a particular patient or patient population; the time of administration, rate of absorption, and rate of excretion; the potential interactions with other drugs taken separately or in combination; and the severity of the particular disease or condition for which a therapeutic effect is desired. The size of the dose will also be determined by the existence, nature, and extent of any adverse side effects that might accompany the administration of a particular compound. Other factors, which affect the specific dosage, include, for example, bioavailability, metabolic profile, and the pharmacodynamics associated with the particular compound to be administered in a particular patient.

For example, a pharmaceutically effective amount can include the amount or quantity of VX-950, which is sufficient to elicit the required or desired therapeutic response, e.g., an amount, which is sufficient to elicit a biological or therapeutic response when administered to a patient.

In some embodiments of this invention, VX-950, or a pharmaceutically acceptable salt thereof, alone or in a spray-dried dispersion, per administration is in an amount of about 250 mg to about 2250 mg. In some embodiments of this invention, VX-950, or a pharmaceutically acceptable salt thereof, per administration is in an amount of about 300 mg to about 1500 mg. In some embodiments of this invention, VX-950, or a pharmaceutically acceptable salt thereof, per administration is in an amount of about 250 mg to about 1250 mg.

In certain embodiments, the dose of VX-950 per administration is at least about 250 mg. In certain embodiments, the dose of VX-950 per administration is at least about 300 mg. In other embodiments, the dose of VX-950 per administration is at least about 400 mg. In other embodiments, the dose of VX-950 per administration is at least about 450 mg. In other embodiments, the dose of VX-950 per administration is at least about 500 mg. In other embodiments, the dose of VX-950 per administration is at least about 600 mg. In other embodiments, the dose of VX-950 per administration is at least about 650 mg. In other embodiments, the dose of VX-950 per administration is at least about 750 mg. In other embodiments, the dose of VX-950 per administration is at least about 850 mg. In other embodiments, the dose of VX-950 per administration is at least about 1000 mg. In other embodiments, the dose of VX-950 per administration is at least about 1125 mg. In other embodiments, the dose of VX-950 per administration is at least about 1250 mg. In other embodiments, the dose of VX-950 per administration is at least about 1500 mg.

In yet other embodiments, the dose of VX-950 per administration is no more than about 1500 mg. In other embodiments, the dose of VX-950 per administration is no more than about 1250 mg. In other embodiments, the dose of VX-950 per administration is no more than about 1125 mg. In other embodiments, the dose of VX-950 per administration is no more than about 1000 mg. In other embodiments, the dose of VX-950 per administration is no more than about 850 mg. In other embodiments, the dose of VX-950 per administration is no more than about 750 mg. In other embodiments, the dose of VX-950 per administration is no more than about 650 mg. In other embodiments, the dose of VX-950 per administration is no more than about 600 mg. In other embodiments, the dose of VX-950 per administration is no more than about 500 mg. In other embodiments, the dose of VX-950 per administration is no more than about 450 mg. In other embodiments, the dose of VX-950 per administration is no more than about 400 mg. In other embodiments, the dose of VX-950 per administration is no more than about 300 mg. In other embodiments, the dose of VX-950 per administration is no more than about 250 mg.

It should be understood that these lower and upper amounts may be combined to provide preferred dose ranges for administering VX-950. For example, in one embodiment, the VX-950, or the pharmaceutically acceptable salt thereof, per administration is in an amount of about 250 mg to about 2250 mg.

In some embodiments, the pharmaceutical formulation is administered in an amount of 10-20 mg VX-950 per administration per kg of body weight. In some embodiments, the pharmaceutical formulation is administered in an amount of 14-18 mg VX-950 per administration per kg of body weight. In some embodiments, the pharmaceutical formulation is administered: a) in an amount of 14 mg VX-950 per kg of body weight; b) in an amount of 16 mg VX-950 per kg of body weight; or c) in an amount of 18 mg VX-950 per kg of body weight.

In any of these embodiments, the amount of VX-950 is administered once a day. Alternatively, the amount of VX-950 is administered twice a day (e.g., BID; every 12 hours (q12h)). Alternatively, the amount of VX-950 is administered three-times per day (e.g., TID; every 8 hours (q8h)). VX-950 may be administered with or without food.

As would be recognized, it advantageous to have flexible dosing schedules. Accordingly, in some embodiments of this invention, the administration is 3 times per day, but not every 8 hours, or twice per day, but not every 12 hours.

In some embodiments, VX-950 is administered to a patient infected with HCV, such that the level of viral RNA in the patient is decreased to undetectable levels and remains at undetectable levels until a “sustained viral response” is achieved. As used herein, “sustained viral response” or “SVR” means that after dosing is completed, viral RNA levels remain undetectable.

This invention also provides a method for providing VX-950 to a human in need thereof, comprising administration to the human an oral dose of a composition comprising VX-950, wherein said dose provides to said human an average plasma concentration (C_{avg}) of VX-950 of at least about 250 ng/mL after the administration. In certain embodiments, the (C_{avg}) is about 1000 ng/mL, about 250 ng/ml, about 300 ng/ml, about 400 ng/ml, about 450 ng/ml, about 500 ng/ml, about 600 ng/ml, about 650 ng/ml, about 750 ng/ml, about 850 ng/ml, about 1000 ng/ml, about 1125 ng/ml or about 1250 ng/ml. In certain embodiments, the (C_{avg}) is obtained/attained within 3 hours after administration, preferably 2 hours, more preferably 1 hour after administering. In a preferred form of these embodiments, the (C_{avg}) is maintained over about 24 hours, and preferably over 12 weeks.

Methods of this invention may also involve administration of another component comprising an additional agent selected from an immunomodulatory agent; an antiviral agent; an inhibitor of HCV protease (other than VX-950); an inhibitor of another target in the HCV life cycle (other than NS3/4A protease); an inhibitor of internal ribosome entry, a broad-spectrum viral inhibitor; or combinations thereof. The additional agent is also selected from an inhibitor of viral cellular entry.

Such anti-viral agents include, but are not limited to, immunomodulatory agents, such as α-, β-, and γ-interferons or thymosin, pegylated derivatized interferon-α compounds, and thymosin; other anti-viral agents, such as ribavirin, amantadine, and telbivudine; other inhibitors of hepatitis proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

Other agents (e.g., non-immunomodulatory or immunomodulatory compounds) may be used in combination with a compound of this invention include, but are not limited to, those specified in WO 02/18369, which is incorporated herein by reference (see, e.g., page 273, lines 9-22 and page 274, line 4 to page 276, line 11 this disclosure being specifically incorporated herein by reference). Still other agents include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosures from those publications are referred to by reference to the publication number, but it should be noted that the disclosure of the compounds in particular is specifically incorporated herein by reference. Examples of such publications include U.S. Patent Application Publication Nos. US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

Still other agents include, but are not limited to, Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.); COPEGUS® (Hoffmann-La Roche, Nutley, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON®(Schering Plough, Interferon-alpha 2B+Ribavirin); pegylated interferon alpha (Reddy, K. R. et al., “Efficacy and Safety of Pegylated (40-kd) Interferon alpha-2a Compared with Interferon alpha-2a in Noncirrhotic Patients with Chronic Hepatitis C,” Hepatology, 33, 433-438 (2001); consensus interferon (INFERGEN®)(Kao, J. H., et al., “Efficacy of Consensus Interferon in the Treatment of Chronic Hepatitis,” J. Gastroenterol. Hepatol., 15, 1418-1423 (2000); lymphoblastoid or “natural” interferon; interferon tau (Clayette, P. et al., “IFN-tau, A New Interferon Type I with Antiretroviral activity” Pathol. Biol. (Paris) 47, 553-559 (1999); interleukin-2 (Davis, G. L. et al., “Future Options for the Management of Hepatitis C.” Seminars in Liver Disease, 19, 103-112 (1999); Interleukin-6 (Davis et al., “Future Options for the Management of Hepatitis C,” Seminars in Liver Disease, 19, 103-112 (1999); interleukin-12 (Davis, G. L. et al., “Future Options for the Management of Hepatitis C.” Seminars in Liver Disease, 19, 103-112 (1999); and compounds that enhance the development of type 1 helper T cell response (Davis et al., “Future Options for the Management of Hepatitis C,” Seminars in Liver Disease, 19, 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod,” J. Am. Acad. Dermatol., 43 S6-11 (2000). See also, WO 02/18369, particularly page 272, line 15 to page 273, line 8, this disclosure being specifically incorporated herein by reference.

As is recognized by skilled practitioners, VX-950 is preferably administered orally. Interferon is not typically administered orally, although orally administered forms are in development. Nevertheless, nothing herein limits the methods or combinations of this invention to any specific dosage forms or regime. Thus, each component of a combination according to this invention may be administered separately, together, or in any combination thereof. As recognized by skilled practitioners, dosages of interferon are typically measured in IU (e.g., about 4 million IU to about 12 million IU). Interferon may also be dosed by micrograms. For example, a standard dose of Peg-Intron is 1.0-1.5 μg/kg/wk and of Pegasys is 180 μg/wk.

Typical peg-IFN and RBV treatment regimens include 12 weeks, 24 weeks, 36 weeks and 48 weeks treatments. Various types of peg-IFN are commercially available, for example, in vials as a prepared, premeasured solution or as a lyophilized (freeze-dried) powder with a separate diluent (mixing fluid). Pegylated interferon alfa-2b (Peg-Intron®) and alfa-2a (Pegasys®) are typical examples. Various types of interferon, including various dosage forms and formulation types, that can be employed in the invention are commercially available (see, e.g., specific examples of interferon described above). For example, various types of interferon are commercially available in vials as a prepared, premeasured solution or as a lyophilized (freeze-dried) powder with a separate diluent (mixing fluid). Pegylated interferon alfa-2b (Peg-Intron®) and alfa-2a (Pegasys®) vary from the other interferons by having molecules of polyethylene glycol (PEG) attached to them. The PEG is believed to cause the interferon to remain in the body longer and thus prolongs the effects of the interferon as well as its effectiveness. Pegylated interferon is generally administered by injection under the skin (subcutaneous). Pegasys® comes as an injectable solution in pre-filled syringes or in vials. The usual dose of Pegasys® is 180 μg, taken once a week. PEG-Intron® generally comes in a pre-filled pen that contains powder and sterile water; pushing down on the pen mixes them together. The dose of PEG-Intron® generally depends on weight-1.5 μg per kilogram (a range of between about 50 and about 150 μg total), taken once a week. In some embodiments, the dose of peg-interferon-alpha-2a is 180 mg/1.73 m², taken subcutaneously once a week. In certain embodiments, a pegylated interferon, e.g., pegylated interferon-alpha 2a or pegylated interfero-alpha 2b, is employed in the invention. Typically, interferon can be dosed according to the dosage regimens described in its commercial product labels.

Ribavirin is typically administered orally, and tablet forms of ribavirin are currently commercially available. General standard, daily dose of ribavirin tablets (e.g., about 200 mg tablets administered twice a day) is about 800 mg to about 1200 mg (according to the dosage regimens described in its commercial product labels). In some embodiments, the dose of ribavirin will be 15 mg/kg/day divided twice daily (capsule or solution) with a maximum of 1,200 mg if weight is ≧75 kg or 1,000 mg if <75 kg.

The methods herein may involve administration or co-administration to a patient a) combinations of VX-950 and another agent; or b) VX-950 in more than one dosage form. Co-administration includes administering each inhibitor in the same dosage form or in different dosage forms. When administered in different dosage forms, the inhibitors may be administered at different times, including about simultaneously or in any time period around administration of the other dosage forms. Separate dosage forms may be administered in any order. That is, any dosage forms may be administered prior to, together with, or following the other dosage forms.

In some aspects, the method includes the administration of agents to a patient over two phases, an initial phase and a secondary phase. For instance the initial phase can be a period of less than about 12 or 24 weeks and the secondary phase can be greater or equal to about 12 weeks, e.g., the secondary phase can be between about 12-36 weeks. In certain embodiments, the initial phase is 12 weeks. In certain embodiments, the initial phase is 24 weeks. In certain embodiments, the secondary phase is 12 weeks. In certain embodiments, the secondary phase is 24 weeks. In still other embodiments, the secondary phase is 36 weeks. In certain embodiments, the sum of the initial and secondary phase is about 24 to 48 weeks (such as 24, 36, or 48 weeks). In some embodiments, the initial and secondary phases can be identical in duration.

VX-950 may be administered in either the initial, secondary, or both phases. In some embodiments, VX-950 is administered only in the initial phase. When VX-950 is administered only in the initial phase, VX-950 may be administered alone or in combination with other agents and one or more agents are administered in the secondary phase. The other agents can be one or more anti-viral agents, one or more other agents described herein, or combinations thereof. In some embodiments, the specific agents administered in the initial and secondary phases are identical.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack comprising at least VX-950 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more of additional agent as described herein. The additional agent or agents may be provided in the same pack or in separate packs.

Another aspect of this involves a packaged kit for a patient to use in the treatment of HCV infection or in the prevention of HCV infection (or for use in another method of this invention), comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection.

Accordingly, this invention provides kits for the simultaneous or sequential administration of a dose of VX-950 (and optionally an additional agent). Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration.

In some embodiments, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

### Example 1

Telaprevir, HPMCAS, and SLS (49.5/49.5/1 w/w/w or 60/39/1 w/w/w or 70/29/1 w/w/w) are solubilized in solvent mixture containing methylene chloride/acetone/water (75/24/1 w/w/w). The solution is spray-dried to render the drug substance amorphous. The spray-dried intermediate is dried during secondary drying to remove residual solvents.

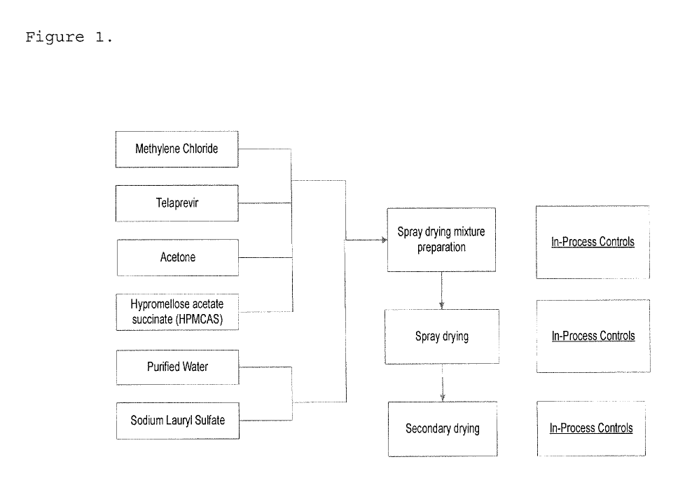

The flow diagram for the manufacturing of telaprevir spray-dried dispersion is shown in FIG. 1.

### Example 2

Telaprevir is supplied as a 562.5 mg tablet for oral administration. The drug product manufacturing process consists of 2 stages. In the first stage, telaprevir drug substance is spray dried with a polymer, hypromellose acetate succinate (HPMCAS), and a surfactant, sodium lauryl sulfate (SLS), to produce an amorphous spray-dried dispersion (SDD) drug product intermediate. In the second stage, the amorphous SDD is blended with additional excipients and compressed into a tablet.

An example of the process of spray-dried dispersion can be found in International Publication Nos. WO 05/123076 and WO 07/109,605, which are incorporated herein by reference. Composition of VX-950 SDD's are provided in Tables 2, 3 and 4.

TABLE 2
Composition of Telaprevir 49.5% Spray-dried Dispersion

| | | Amount | |
|---|---|---|---|
| | Component | Per 1000 | Content |
| Component | Function | mg | (% w/w) |
| Telaprevir drug substance | API | 495 | 49.5 |
| Hypromellose acetate | Physical | 495 | 49.5 |
| succinate-HPMCAS HG | stabilizer | | |
| Sodium lauryl sulfate (SLS) | Wetting agent | 10 | 1 |
| Methylene chloride^{a} | Process solvent | — | — |
| Acetone^{a} | Process solvent | — | — |
| Purified water^{a} | Process solvent | — | — |
| Total | | 1000 | 100 |

TABLE 3
Composition of Telaprevir Spray-dried Dispersion, 60% M

| | | Amount | |
|---|---|---|---|
| | Component | Per 1000 | Content |
| Component | Function | mg | (% w/w) |
| Telaprevir drug substance | API | 600 | 60 |
| Hypromellose acetate | Physical | 390 | 39 |
| succinate-HPMCAS MG | stabilizer | | |
| Sodium lauryl sulfate (SLS) | Wetting agent | 10 | 1 |
| Methylene chloride^{a} | Process solvent | — | — |
| Acetone^{a} | Process solvent | — | — |
| Purified water^{a} | Process solvent | — | — |
| Total | | 1000 | 100 |

^{a}Removed during processing

TABLE 4
Composition of Telaprevir Spray-dried Dispersion, 70% M

| | | Amount | |
|---|---|---|---|
| | Component | Per 1000 | Content |
| Component | Function | mg | (% w/w) |
| Telaprevir drug substance | API | 700 | 70 |
| Hypromellose acetate | Physical | 290 | 29 |
| succinate-HPMCAS MG | stabilizer | | |
| Sodium lauryl sulfate (SLS) | Wetting agent | 10 | 1 |
| Methylene chloride^{a} | Process solvent | — | — |
| Acetone^{a} | Process solvent | — | — |
| Purified water^{a} | Process solvent | — | — |
| Total | | 1000 | 100 |

^{a}Removed during processing

Composition of VX-950 Tablets are provided in Tables 5, 6 and 7.

TABLE 5
Composition of Telaprevir 49.5% H + 70% M tablet, 562.5 mg

| | | Amount | |
|---|---|---|---|
| | | per | Content |
| | Component | Tablet | (% |
| Component | Function | (mg) | w/w) |
| Telaprevir from 49.5% Spray-dried | API | 233 | 21.06 |
| Dispersion | | | |
| Hypromellose acetate succinate- | Physical | 233 | 21.06 |
| HPMCAS HG from 49.5% | stabilizer | | |
| Spray-dried Dispersion | | | |
| Sodium lauryl sulfate (SLS) from | Wetting | 4.71 | 0.43 |
| 49.5% Spray-dried Dispersion | agent | | |
| Telaprevir from 70% Spray-dried | API | 329.5 | 29.78 |
| Dispersion | | | |
| Hypromellose acetate succinate- | Physical | 136.5 | 12.34 |
| HPMCAS MG from 70% | stabilizer | | |
| Spray-dried Dispersion | | | |
| Sodium lauryl sulfate (SLS) | Wetting | 4.71 | 0.43 |
| from 70% Spray-dried Dispersion | agent | | |
| Microcrystalline cellulose | Diluent, | 37.7 | 3.40 |
| | Disintegrant | | |
| Dibasic calcium phosphate, anhydrous | Diluent | 37.7 | 3.40 |
| Croscarmellose sodium | Disintegrant | 47.1 | 4.25 |
| Colloidal silicon dioxide | Flow agent | 9.4 | 0.85 |
| Sodium stearyl fumarate | Lubricant | 33.2 | 3.00 |
| Total | — | 1106.41 | 100.00 |

TABLE 6
Composition of Telaprevir 60% M Tablet, 562.5 mg

| | | Amount | |
|---|---|---|---|
| | | per | Content |
| | Component | Tablet | (% |
| Component | Function | (mg) | w/w) |
| Telaprevir from 60% Spray-dried | API | 562.5 | 51.05 |
| Dispersion | | | |
| Hypromellose acetate succinate- | Physical | 365.63 | 33.18 |
| HPMCAS MG from 60% | stabilizer | | |
| Spray-dried Dispersion | | | |
| Sodium lauryl sulfate (SLS) from | Wetting | 9.38 | 0.85 |
| 60% Spray-dried Dispersion | agent | | |
| Microcrystalline cellulose | Diluent, | 37.5 | 3.40 |
| | Disintegrant | | |
| Dibasic calcium phosphate, anhydrous | Diluent | 37.5 | 3.40 |
| Croscarmellose sodium | Disintegrant | 46.9 | 4.25 |
| Colloidal silicon dioxide | Flow agent | 9.4 | 0.85 |
| Sodium stearyl fumarate | Lubricant | 33.1 | 3.00 |
| Total | — | 1101.80 | 100.00 |

TABLE 7
Composition of Composition of Telaprevir
70% M Tablet, 562.5 mg

| | | Amount | |
|---|---|---|---|
| | | per | Content |
| | Component | Tablet | (% |
| Component | Function | (mg) | w/w) |
| Telaprevir from 70% Spray-dried | API | 562.5 | 59.56 |
| Dispersion | | | |
| Hypromellose acetate succinate- | Physical | 233.0 | 24.68 |
| HPMCAS MG from 70% | stabilizer | | |
| Spray-dried Dispersion | | | |
| Sodium lauryl sulfate (SLS) from | Wetting | 8.04 | 0.85 |
| 70% Spray-dried Dispersion | agent | | |
| Microcrystalline cellulose | Diluent, | 32.1 | 3.40 |
| | Disintegrant | | |
| Dibasic calcium phosphate, anhydrous | Diluent | 32.1 | 3.40 |
| Croscarmellose sodium | Disintegrant | 40.2 | 4.25 |
| Colloidal silicon dioxide | Flow agent | 8.0 | 0.85 |
| Sodium stearyl fumarate | Lubricant | 28.3 | 3.00 |
| Total | — | 944.40 | 100.00 |

### Example 3

Process Description for telaprevir 60% M tablet, 562.5 mg and telaprevir 70% M tablet, 562.5 mg

Blending:

The spray dried dispersion composed of Telaprevir:HPMCAS MG:SLS and excipients are individually weighted out in the required amounts for the batch manufacture. The excipients are Microcrystalline cellulose (Avicel PH113), Dibasic calcium phosphate anhydrous (ATAB), Croscarmellose sodium (AcDiSol), Colloidal silicon dioxide (Cabosil) and Sodium stearyl fumarate (SSF). The API and excipients (except SSF) are screened through a sieve and dispensed into blender of appropriate size. Upon dispensing, the API and excipients are blended to achieve batch homogeneity. The excipient SSF is separately screened through a sieve. Upon completion of initial blending step, screened SSF is added directly into the blender. The batch is then blended with lubricant SSF for appropriate amount of time and number of revolutions.

Compression:

The tablet compression is performed in the room under controlled relative humidity (˜30%).

The tablet press is tooled with appropriate tool punches and dies prior to the onset of compression. After transferring the blend into hopper attached to tablet press, the press is started in automatic mode that produces tablets of specified weights and hardnesses.

Bulk Packaging:

The tablets are packaged inside a LDPE bag (primary container). The LDPE bags are packaged inside a heat sealed aluminum foil bags with molecular sieve desiccant (4-6% by weight of tablets) in between bags.

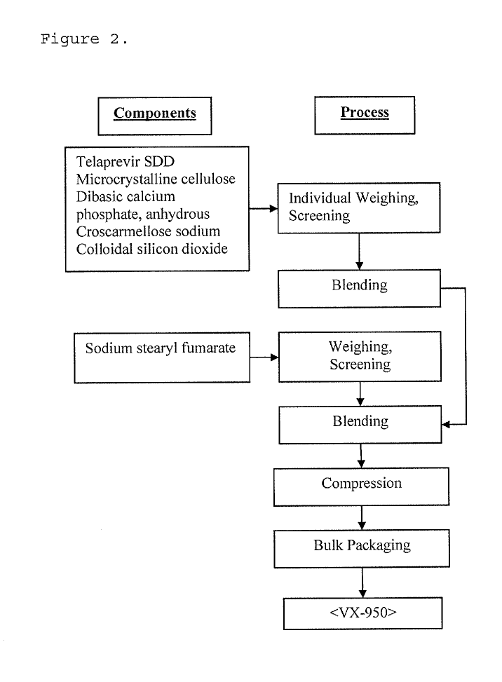

The Manufacturing Process Flow Diagram for Telaprevir 60% M tablet, 562.5 mg and Telaprevir 70% M tablet, 562.5 mg is shown in FIG. 2.

### Example 4

Process Description for telaprevir 49.5% H+70% M tablet, 562.5 mg

Blending:

The spray dried dispersion composed of 49.5% Telaprevir: 49.5% HPMCAS H: 1% SLS, spray dried dispersion composed of 70% Telaprevir: 29% HPMCAS MG: 1% SLS and excipients are individually weighted out in the required amounts for the batch manufacture. The excipients are Microcrystalline cellulose (Avicel PH113), Dibasic calcium phosphate anhydrous (ATAB), Croscarmellose sodium (AcDiSol), Colloidal silicon dioxide (Cabosil) and Sodium stearyl fumarate (SSF). Both SDDs and excipients (except SSF) are screened through a sieve and dispensed into blender of appropriate size. Upon dispensing, the API and excipients are blended to achieve batch homogeneity. The excipient SSF is separately screened through a sieve. Upon completion of initial blending step, screened SSF is added directly into the blender. The batch is then blended with SSF (lubricant) for appropriate amount of time and number of revolutions.

Compression:

The tablet compression is performed in the room under controlled relative humidity (−30%).

The tablet press is tooled with appropriate tool punches and dies prior to the onset of compression. After transferring the blend into hopper attached to tablet press, the press is started in automatic mode that produces tablets of specified weights and hardnesses.

Bulk Packaging:

The tablets are packaged inside a LDPE bag (primary container). The LDPE bags are packaged inside a heat sealed aluminum foil bags with molecular sieve desiccant (4-6% by weight of tablets) in between bags.

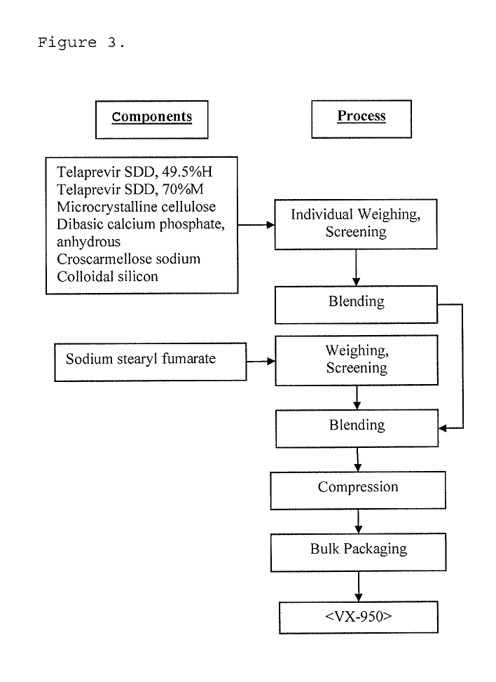

The Manufacturing Process Flow Diagram for Telaprevir 49.5% H+70% M tablet, 562.5 mg is shown in FIG. 3.

### Example 5

Description of the manufacturing process for 60% M VX950 Spray Dried Dispersion.

The amount of each component required is described in Table 8 below.

Charge Methylene Chloride (DCM) into the main solvent vessel.

Set the vessel temperature to 2° C.-8° C., preferably 5° C.

Charge VX-950 drug substance into the main solvent vessel.

Verify that the drug substance is dissolved.

Add acetone to the main solvent vessel after the VX-950 is fully dissolved.

Charge HPMCAS-MG into the main solvent vessel.

Verify that all solids are dissolved.

In secondary mixing vessel charge purified water.

Add SLS to purified water into the secondary mixing vessel.

Verify that SLS is dissolved.

Add SLS and water pre-mixed solution to the main mixing vessel and verify that all components in the resultant mixture are dissolved.

Spray dry the resultant mixture solution from the main mixing vessel using the Spraying Systems SK pressure nozzle (70-16) with the spray dryer being inertized with nitrogen and heated to the appropriate outlet temperature.

Wet spray-dried dispersion (SDD) particles are inertially separated from the process gas by a cyclone and collected within collection chamber. The process gas is then filtered for fine particles and passed through a condenser to remove process solvent

Wet SDD is dried in a convection tray oven with a temperature profile of 1 hour ramp to 40° C., hold at 40° C. for three hours, 1 hour ramp to 60° C., and hold at 60° C. for 10 hours.

Package dried SDD is stored in LDPE bags and heat seal in Al foil bags with desiccant in between and keep at ambient temperature if residual solvents (methylene chloride, acetone, toluene, ethyl acetate) are below the established specifications.

TABLE 8
Formulation composition of the 60% API spray dried dispersion based
on 16 kg total SDD at 15 wt % solids load for spray drying.

| Component | | % | mass |
|---|---|---|---|
| Function | Component | (w/w) | [kg] |
| API | VX-950 | 9.00 | 9.60 |
| Polymer | Hypromellose Acetate Succinate, | 5.85 | 6.24 |
| (Dispersant) | (HPMCAS-MG, Shin-Etsu) | | |
| Surfactant | Sodium Lauryl Sulfate (SLS) | 0.15 | 0.16 |
| Process | Methylene Chloride (for Dispersion) ^{a} | 63.75 | 68.00 |
| Solvent | | | |
| Process | Acetone (for Dispersion) ^{a} | 20.4 | 21.76 |
| Solvent | | | |
| Process | Purified Water (for Dispersion) ^{a} | 0.85 | 0.907 |
| Solvent | | | |

^{a} Removed during processing

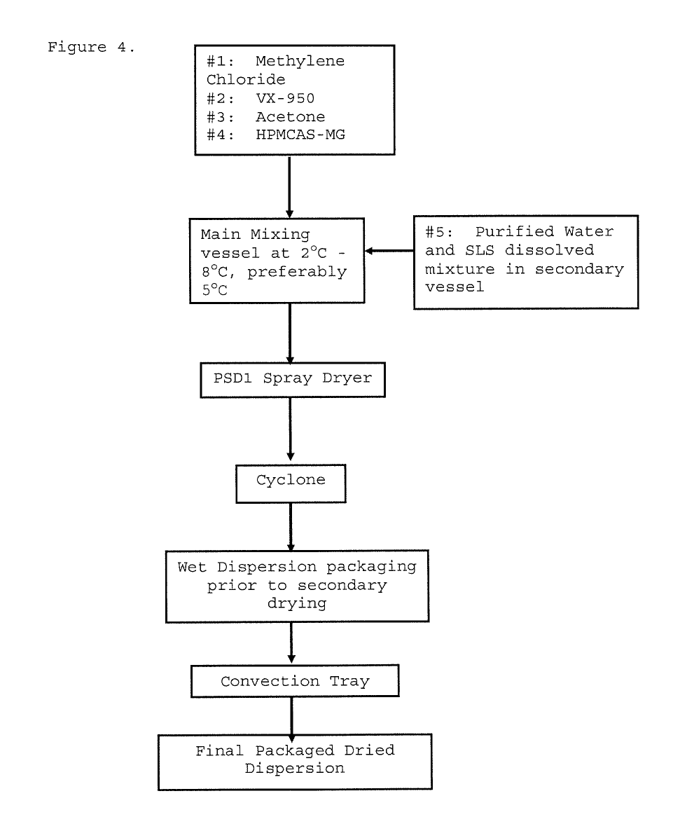

The Manufacturing Process Flow Diagram for telaprevir 60% M VX-950 Spray Dried Dispersion is shown in FIG. 4.

### Example 6

Description of Manufacturing Process for 60% M VX950 Spray Dried Dispersion.

The amount of each component required is described in Table 9 below.

Prepare Methylene Chloride in the equilibration solvent tank.

Charge SLS into the secondary mixing vessel Charge purified water into a secondary mixing vessel.

Dissolve SLS fully within the purified water.

Charge about half the total amount of Methylene Chloride to the main solution reactor.

The reactor is set to 2-8° C., preferably 5° C. Charge VX-950 drug substance into the main solution reactor.

Add the remaining quantities of Methylene Chloride to the main solution reactor

and verify that the drug substance is dissolved.

After the VX-950 is fully dissolved, charge the HPMCAS-MG into the main solution reactor.

Add the acetone amount to the mixing reactor. and verify all solids are dissolved.

Add the SLS and water pre-mixed solution to the main mixing reactor and verify that all components in the resultant mixture have been dissolved.

Spray dry the resultant mixture solution from the main mixing reactor with the spray dryer being inertized with nitrogen heated to the appropriate outlet temperature.

Wet SDD particles are inertially separated from the process gas by a cyclone and collected within stainless steel intermediate bulk containers. The process gas is then filtered for fine particles and passed through a condenser to remove process solvents.

Wet SDD is charged and dried in the secondary dryer until all residual solvents (methylene chloride and acetone) are below the specifications established, and preferably each below 200 ppm.

Stabilize the dispersions to 20-30° C. prior to packaging.

Sieve secondary-dried SDD using a number 16 mesh in nitrogen inertized glovebox. The sieved SDD is then packaged in a double LDPE bags within an aluminum laminate bag with molecular sieve desiccant (5% of the solid dispersion) placed in between LDPE bags and Al foil bag.

TABLE 9
Formulation composition of 60%/39%/1% wt/wt/wt
VX-950/HPMCAS-MG/SLS formulation in 75%/24%/1%
wt/wt/wt DCM:Acetone:Water solvent system.

| Component | | % | Weight |
|---|---|---|---|
| Function | Component | Weight | (kg) |
| API | VX-950 | 9.00 | 180 |
| Polymer | Hydroxypropyl Methylcellulose | 5.85 | 117 |
| | Acetate Succinate, USP/JPE | | |
| | (HPMCAS-MG) | | |
| Surfactant | Sodium Lauryl Sulfate, NF | 0.15 | 3 |
| | (SLS) | | |
| Process | Methylene Chloride, NF ^{a} | 63.75 | 1275 |
| Solvent | | | |
| Process | Acetone, NF ^{a} | 20.40 | 408 |
| Solvent | | | |
| Process | Water, USP ^{a} | 0.85 | 17 |
| Solvent | | | |
| Total | — | 100 | 2000 |

^{a} Removed during processing

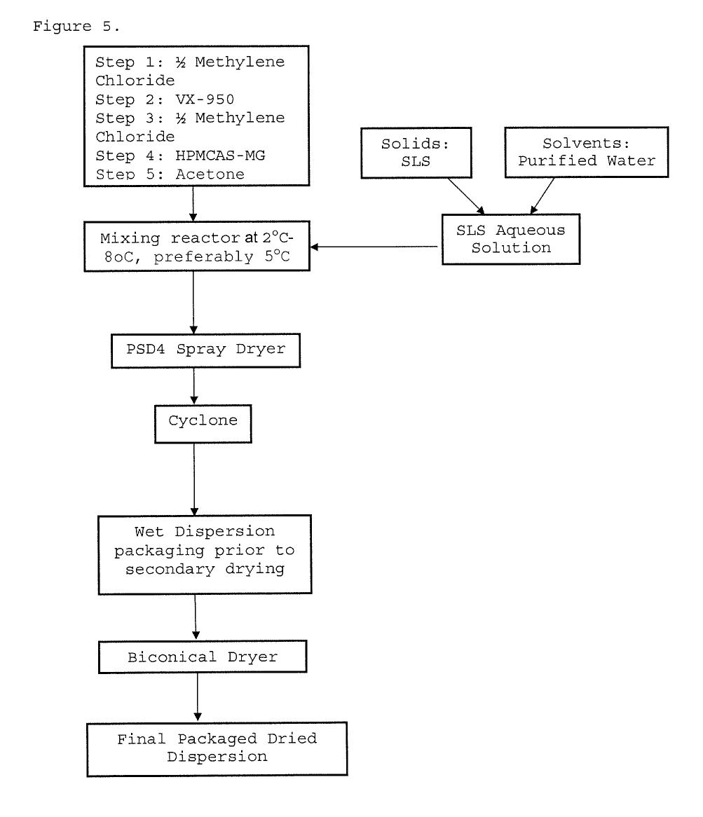

The Manufacturing Process Flow Diagram for telaprevir 60% M Spray Dried Dispersion is shown in FIG. 5.

### Example 7

Description of Manufacturing Process for 60% M VX950 tablet, 562.5 mg.

The amount of each component required is described in Table 11 below.

Weigh the required amount of VX-950 solid dispersion, A-Tab, Avicel PH113, Ac-Di-Sol, Cabosil and Sodium Stearyl Fumarate (SSF) according to the amount described in Table 11 below.

Screen Cabosil and A-Tab simultaneously through a Sweco equipped with a 30M mesh screen or hand screen through a #30 mesh screen into a container.

Screen Avicel PH113 and Ac-Di-Sol in that order through a Sweco equipped with a 30M mesh screen or hand screen through a #30 mesh screen into a container.

Pass the VX-950 solid dispersion through a Sweco equipped with 30 mesh screen into a container or hand screen through a #30 mesh screen into a container.

Set up 600 L bin blender and transfer the above pre-screened materials into the bin blender.

Blend the materials in bin blender for 72±10 revolutions (approx. 9 minutes) at fixed speed (typically 8 rpm).

Screen SSF through a Sweco equipped with a 60M screen or #60 mesh hand screen into a container.

Add the screened SSF to the mixture in the bin blender. Blend in the bin blender for 72±10 revolutions (−9 minutes) at fixed speed (typically 8 rpm). Transfer the lubricated blend from the bin blender for compression using a 12 station Korsch XM12 Press at the press speed of 15-25 RPM.

Compressed tablets have the weight and hardness as listed in Table 10.

TABLE 10
Target and Acceptable Ranges for Tablet Weight and Hardness

| Tablet Properties | Values |
|---|---|
| Average weight (mg) | 1102 |
| Target Range (±3%) | 1068-1135 |
| Acceptable Range (±5%) | 1046-1157 |
| Individual weight (mg) | 1102 |
| Target Range (±3%) | 1068-1135 |
| Acceptable Range (±5%) | 1046-1157 |
| Hardness (kP) | 25 |
| Individual Target Range | 22-28 |
| (kP) | |
| Individual Acceptable | 20-30 |
| Range (kP) | |

TABLE 11
Formulation Composition of VX-950 tablets

| | % | Weight |
|---|---|---|
| Material Name | W/W | (kg) |
| VX-950 SDD (60% VX-950, 39% | 85.10 | 123.4 |
| HPMCAS-MG, 1% SLS) | | |
| Microcrystalline Cellulose, | 3.40 | 4.93 |
| NF Avicel PH113 | | |
| A-Tab (Dical phosphate | 3.40 | 4.93 |
| Anhydrous) | | |
| Croscarmellose Sodium, NF | 4.25 | 6.16 |
| (Ac-Di-Sol) | | |
| Ca-O-Sil (Fumed Silicon | 0.85 | 1.23 |
| Dioxide) | | |
| Sodium Stearyl Fumarate NF | 3.00 | 4.35 |
| Total | 100.00 | 145 |

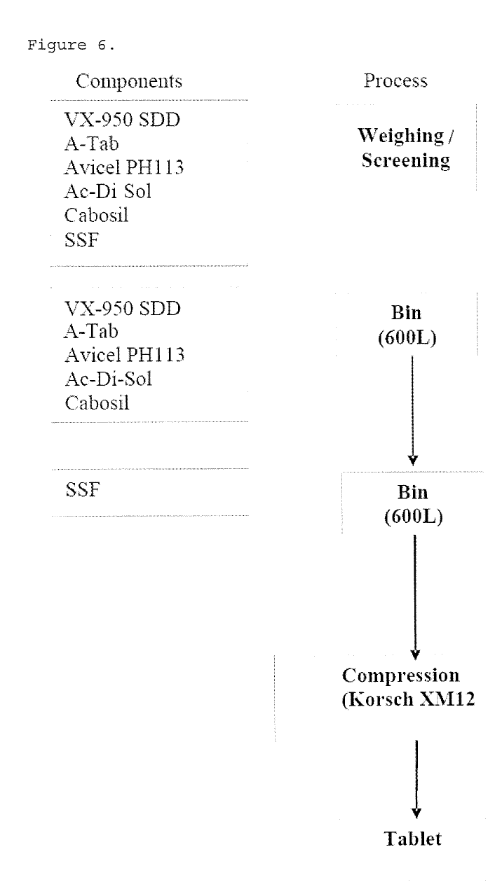

The Manufacturing Process Flow Diagram for telaprevir 60% M tablet, 562.5 mg, is shown in FIG. 6.

### Example 8

In a phase 1, randomized, open-label, cross-over design in 36 healthy male subjects, formulations with different drug loads of VX-950 were evaluated. Subjects were administered a single oral dose of 750 mg of VX-950 in the fed state.

TABLE 12
Mean plasma concentration in ng/mL (percent of coefficient
variation) of VX-950 formulations with different drug loads

| Time | 60% VX-950/39% | 49.5% VX-950/49.5% |
|---|---|---|
| (hr) | HPMCAS-H/1% SLS | HPMCAS-H/1% SLS |
| 0.00 | 0.00(—) | 0.00(—) |
| 0.25 | 0.21(346.41) | 0.85(151.31) |
| 0.50 | 5.47(122.32) | 4.22(124.24) |
| 0.75 | 26.40(143.98) | 11.69(118.33) |
| 1.00 | 54.15(133.15) | 37.60(100.49) |
| 2.00 | 346.66(105.93) | 549.81(95.22) |
| 2.50 | 600.26(90.13) | 1116.33(78.47) |
| 3.00 | 961.83(84.42) | 1591.58(61.59) |
| 3.50 | 1272.75(67.89) | 2058.08(59.98) |
| 4.00 | 1443.50(53.58) | 2299.33(45.18) |
| 6.00 | 1232.92(45.12) | 1698.67(36.02) |
| 8.00 | 692.42(47.90) | 1008.58(46.86) |
| 10.00 | 503.67(69.73) | 626.25(51.73) |
| 12.00 | 286.23(63.33) | 395.00(56.91) |
| 16.00 | 117.72(76.05) | 162.24(80.97) |
| 24.00 | 36.91(106.88) | 48.58(98.95) |

### Example 9

In a phase 1, randomized, open-label, crossover study the relative bioavailability of novel oral formulations of telaprevir was evaluated. A single 1125-mg dose of VX-950 was administered to healthy subjects in the fed state.

TABLE 13
Mean (percent of coefficient variation)
of VX-950 Pharmacokinetic Parameters

| SDD Formulation | AUC_{0-∞} | Cₘₐₓ |
|---|---|---|
| 60% VX-950/39% HPMCAS-M/1% | 26030.6 (28.4) | 2905.4 (31.4) |
| SLS | | |
| 70% VX-950/29% HPMCAS-M/1% | 12338.3 (48.9) | 1633.1 (46.6) |
| SLS | | |
| 49.5% H and 70% M blend | 20755.1 (36.1) | 2416.6 (30.6) |

### Example 10

One tablet (375 mg dose) of each formulation was dropped into a 75 ml vial containing simulated intestinal fluid (FESSIF D<01> recipe) at pH 5.0 or pH 6.8 and temperature was maintained at 37° C. via heating element. The 75 ml vial was stirred with stir bar ˜130 RPM throughout the experiment. Aliquots for HPLC analysis were taken at predetermined time points.

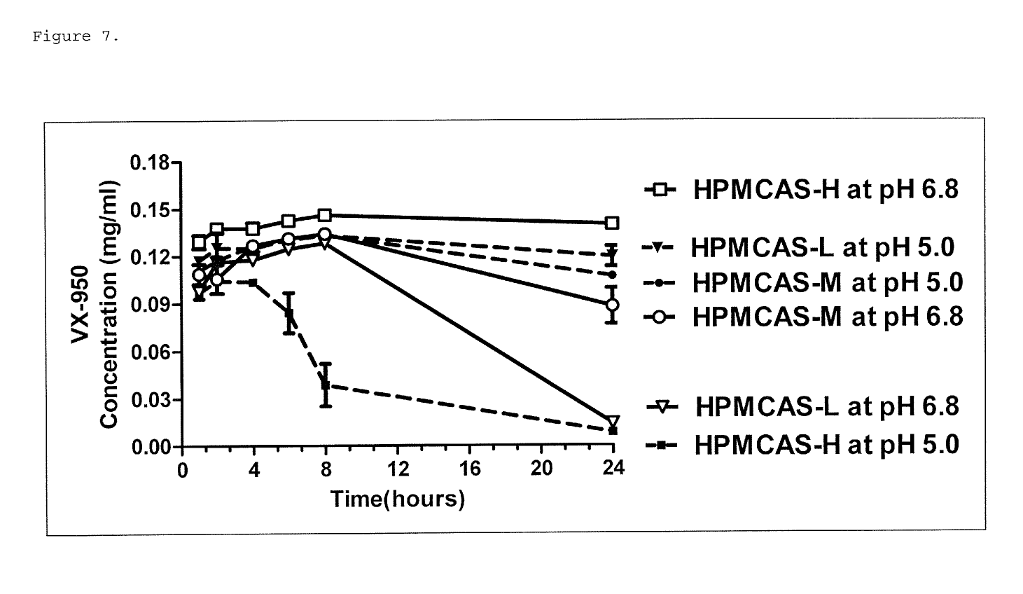

TABLE 14
Comparison of Kinetic Solubility of 60% L, 60% M and 60%
H SDD VX-950 tablets at pH 5.0 and pH 6.8 (FIG. 7).

| Time | | |
|---|---|---|
| (hrs) | | |
| | 60% VX-950 H | 60% VX-950 H |
| | pH 5.0 | pH 6.8 |

| | | | | |
|---|---|---|---|---|
| 1 | 0.094 | 0.099 | 0.132 | 0.126 |
| 2 | 0.103 | 0.105 | 0.139 | 0.136 |
| 4 | 0.104 | 0.103 | 0.140 | 0.135 |
| 6 | 0.093 | 0.075 | 0.144 | 0.141 |
| 8 | 0.048 | 0.029 | 0.148 | 0.144 |
| 24 | 0.008 | 0.009 | 0.142 | 0.138 |

| | |
|---|---|
| 60% VX-950 L | 60% VX-950 L |
| pH 5.0 | pH 6.8 |

| | | | | |
|---|---|---|---|---|
| 1 | 0.115 | 0.119 | 0.097 | 0.098 |
| 2 | 0.118 | 0.132 | 0.117 | 0.115 |
| 4 | 0.123 | 0.126 | 0.118 | 0.117 |
| 6 | 0.129 | 0.132 | 0.124 | 0.125 |
| 8 | 0.131 | 0.135 | 0.128 | 0.128 |
| 24 | 0.115 | 0.124 | 0.012 | 0.016 |

| | |
|---|---|
| 60% VX-950 M | 60% VX-950 M |
| pH 5.0 | pH 6.8 |

| | | | | |
|---|---|---|---|---|
| 1 | 0.109 | 0.112 | 0.104 | 0.113 |
| 2 | 0.119 | 0.115 | 0.099 | 0.112 |
| 4 | 0.127 | 0.126 | 0.124 | 0.129 |
| 6 | 0.129 | 0.131 | 0.130 | 0.132 |
| 8 | 0.133 | 0.133 | 0.135 | 0.133 |
| 24 | 0.107 | 0.107 | 0.080 | 0.096 |

TABLE 15
Kinetic Solubility of 49.5% H and 70% M SDD
blend VX-950 tablets at pH 5.0 and pH 6.8.

| | | |
|---|---|---|
| | 49.5% H and | 49.5% H and |
| Time | 70% M VX-950 | 70% M VX-950 |
| (hrs) | blend pH 5.0 | blend pH 6.8 |

| | | | | |
|---|---|---|---|---|
| 1 | 0.117 | 0.116 | 0.163 | 0.160 |
| 2 | 0.122 | 0.121 | 0.160 | 0.159 |
| 4 | 0.124 | 0.122 | 0.157 | 0.153 |
| 6 | 0.138 | 0.124 | 0.157 | 0.150 |
| 8 | 0.136 | 0.133 | 0.164 | 0.152 |
| 24 | 0.103 | 0.102 | 0.162 | 0.153 |

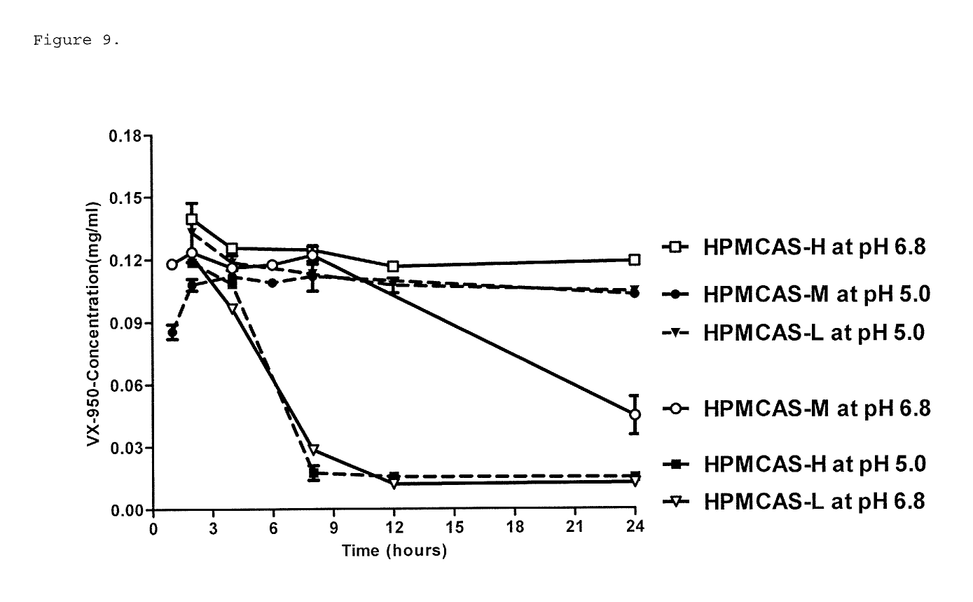

TABLE 16
Kinetic Solubility of 70% L and 70% M SDD VX-
950 tablets at pH 5.0 and pH 6.8 (FIG. 9).

| Time | | |
|---|---|---|
| (hrs) | | |
| | 70% L VX-950 | 70% L VX-950 |
| | pH 5.0 | pH 6.8 |

| | | | | |
|---|---|---|---|---|
| 2 | 0.143 | 0.123 | 0.121 | 0.122 |
| 4 | 0.121 | 0.116 | 0.103 | 0.090 |
| 8 | 0.114 | 0.112 | 0.022 | 0.035 |
| 12 | 0.105 | 0.110 | 0.009 | 0.015 |
| 24 | 0.105 | 0.104 | 0.013 | 0.012 |

| | |
|---|---|
| 70% M VX-950 | 70% M VX-950 |
| pH 5.0 | pH 6.8 |

| | | | | |
|---|---|---|---|---|
| 1 | 0.083 | 0.088 | 0.117 | 0.119 |
| 2 | 0.106 | 0.110 | 0.123 | 0.124 |
| 4 | 0.111 | 0.113 | 0.116 | 0.116 |
| 6 | 0.109 | 0.109 | 0.117 | 0.118 |
| 8 | 0.117 | 0.107 | 0.119 | 0.125 |
| 24 | 0.103 | 0.103 | 0.051 | 0.038 |

| | |
|---|---|
| 70% H VX-950 | 70% H VX-950 |
| pH 5.0 | pH 6.8 |

| | | | | |
|---|---|---|---|---|
| 2 | 0.118 | 0.119 | 0.133 | 0.146 |
| 4 | 0.108 | 0.109 | 0.122 | 0.129 |
| 8 | 0.015 | 0.020 | 0.122 | 0.127 |
| 12 | 0.014 | 0.017 | 0.118 | 0.115 |
| 24 | 0.014 | 0.016 | 0.115 | 0.123 |

We claim:

1. A spray-dried dispersion comprising VX-950, sodium lauryl sulfate and a polymer selected from the group consisting of hypromellose acetate succinate-M, hypromellose acetate succinate-L and hypromellose acetate succinate-H.

2. The spray-dried dispersion of claim 1, wherein the spray-dried dispersion comprises from about 40% to about 80% wt/wt VX-950.

3. The spray-dried dispersion of claim 2, wherein the spray-dried dispersion comprises about 60% wt/wt VX-950.

4. The spray-dried dispersion of claim 2, wherein the spray-dried dispersion comprises about 70% wt/wt VX-950.

5. The spray-dried dispersion of any one of claims 1-4, wherein the spray-dried dispersion comprises from about 20% to about 50% wt/wt hypromellose acetate succinate-M.

6. The spray-dried dispersion of claim 5, wherein the spray-dried dispersion comprises about 29% wt/wt hypromellose acetate succinate-M.

7. The spray-dried dispersion of claim 5, wherein the spray-dried dispersion comprises about 39% wt/wt hypromellose acetate succinate-M.

8. The spray-dried dispersion of any one of claims 1-4, wherein the spray-dried dispersion comprises from about 20% to about 50% wt/wt hypromellose acetate succinate-L.

9. The spray-dried dispersion of claim 8, wherein the spray-dried dispersion comprises about 29% wt/wt hypromellose acetate succinate-L.

10. The spray-dried dispersion of claim 8, wherein the spray-dried dispersion comprises about 39% wt/wt hypromellose acetate succinate-L.

11. The spray-dried dispersion of any one of claims 1-4, wherein the spray-dried dispersion comprises from about 20% to about 60% wt/wt hypromellose acetate succinate-H.

12. The spray-dried dispersion of claim 11, wherein the spray-dried dispersion comprises about 29% wt/wt hypromellose acetate succinate-H.

13. The spray-dried dispersion of claim 11, wherein the spray-dried dispersion comprises about 39% wt/wt hypromellose acetate succinate-H.

14. The spray-dried dispersion of any one of claims 1-13, wherein the spray-dried dispersion comprises about 1% wt/wt sodium lauryl sulfate.

15. The spray-dried dispersion of claim 14, wherein the spray-dried dispersion comprises about 60% wt/wt VX-950, about 39% hypromellose acetate succinate-M and about 1% sodium lauryl sulfate.

16. The spray-dried dispersion of claim 14, wherein the spray-dried dispersion comprises about 70% wt/wt VX-950, about 29% hypromellose acetate succinate-M and about 1% sodium lauryl sulfate.

17. A spray-dried dispersion of VX-950 comprising VX-950, sodium lauryl sulfate and two or more polymers selected from the group consisting of hypromellose acetate succinate-H, hypromellose acetate succinate-M and hypromellose acetate succinate-L.

18. A pharmaceutical composition comprising a spray-dried dispersion of VX-950, the spray dried dispersion comprising VX-950, sodium lauryl sulfate and a polymer selected from the group consisting of hypromellose acetate succinate-H, hypromellose acetate succinate-M and hypromellose acetate succinate-L.

19. The pharmaceutical composition of claim 18, wherein the spray-dried dispersion comprises from about 40% to about 80% wt/wt VX-950.

20. The pharmaceutical composition of claim 19, wherein the spray-dried dispersion comprises about 60% wt/wt VX-950.

21. The pharmaceutical composition of claim 19, wherein the spray-dried dispersion comprises about 70% wt/wt VX-950.

22. The pharmaceutical composition of any one of claims 18-21, wherein the spray-dried dispersion comprises from about 20% to about 50% wt/wt hypromellose acetate succinate-M.

23. The pharmaceutical composition of claim 22, wherein the spray-dried dispersion comprises about 29% wt/wt hypromellose acetate succinate-M.

24. The pharmaceutical composition of claim 22, wherein the spray-dried dispersion comprises about 39% wt/wt hypromellose acetate succinate-M.

25. The pharmaceutical composition of any one of claims 18-21, wherein the spray-dried dispersion comprises from about 20% to about 50% wt/wt hypromellose acetate succinate-L.

26. The pharmaceutical composition of claim 25, wherein the spray-dried dispersion comprises about 29% wt/wt hypromellose acetate succinate-L.

27. The pharmaceutical composition of claim 25, wherein the spray-dried dispersion comprises about 39% wt/wt hypromellose acetate succinate-L.

28. The pharmaceutical composition of any one of claims 18-21, wherein the spray-dried dispersion comprises from about 20% to about 60% wt/wt hypromellose acetate succinate-H.

29. The pharmaceutical composition of claim 28, wherein the spray-dried dispersion comprises about 29% wt/wt hypromellose acetate succinate-H.

30. The pharmaceutical composition of claim 28, wherein the spray-dried dispersion comprises about 39% wt/wt hypromellose acetate succinate-H.

31. The pharmaceutical composition of any one of claims 18-30, wherein the spray-dried dispersion comprises about 1% wt/wt sodium lauryl sulfate.

32. The pharmaceutical composition of claim 31, wherein the spray-dried dispersion comprises about 60% wt/wt VX-950, about 39% hypromellose acetate succinate-M and about 1% sodium lauryl sulfate.

33. The pharmaceutical composition of claim 31, wherein the spray-dried dispersion comprises about 70% wt/wt VX-950, about 29% hypromellose acetate succinate-M and about 1% sodium lauryl sulfate.

34. A pharmaceutical composition comprising a first spray-dried dispersion comprising VX-950, hypromellose acetate succinate-M and sodium lauryl sulfate and a second spray-dried dispersion comprising VX-950, hypromellose acetate succinate-H and sodium lauryl sulfate.

35. A pharmaceutical composition comprising a first spray-dried dispersion comprising VX-950, hypromellose acetate succinate-M and sodium lauryl sulfate and a second spray-dried dispersion comprising VX-950, hypromellose acetate succinate-L and sodium lauryl sulfate.

36. A pharmaceutical composition comprising a first spray-dried dispersion comprising VX-950, hypromellose acetate succinate-L and sodium lauryl sulfate and a second spray-dried dispersion comprising VX-950, hypromellose acetate succinate-H and sodium lauryl sulfate.

37. The pharmaceutical composition of any one of claims 34-36, wherein the first spray-dried dispersion comprises from about 40% to about 80% VX-950.

38. The pharmaceutical composition of any one of claims 34-36, wherein the first spray-dried dispersion comprises about 70% wt/wt VX-950.

39. The pharmaceutical composition of any one of claims 34-36, wherein the first spray-dried dispersion comprises about 60% wt/wt VX-950.

40. The pharmaceutical composition of claim of any one of claims 34-36, wherein the second spray-dried dispersion comprises about 49.5% wt/wt VX-950.

41. The pharmaceutical composition of claim 34 or 35, wherein the first spray-dried dispersion comprises from about 20% to about 50% hypromellose acetate succinate-M.

42. The pharmaceutical composition of claim 36, wherein the first spray-dried dispersion comprises from about 50% to about 80% hypromellose acetate succinate-L.

43. The pharmaceutical composition of claim 35, wherein the second spray-dried dispersion comprises from about 50% to about 80% hypromellose acetate succinate-L.

44. The pharmaceutical composition of claim 34 or 35, wherein the first spray-dried dispersion comprises about 29% hypromellose acetate succinate-M.

45. The pharmaceutical composition of claim 34 or 35, wherein the first spray-dried dispersion comprises about 39% hypromellose acetate succinate-M.

46. The pharmaceutical composition of claim 34 or 36, wherein the second spray-dried dispersion comprises about 49.5% hypromellose acetate succinate-H.

47. The pharmaceutical composition of any one of claims 34-46, wherein the first spray-dried dispersion comprises about 1% sodium lauryl sulfate.

48. The pharmaceutical composition of any one of claims 34-46, wherein the second spray-dried dispersion comprises about 1% sodium lauryl sulfate.

49. The pharmaceutical composition of claim 34 or 35, wherein the first spray-dried dispersion comprises about 70% wt/wt VX-950, about 29% hypromellose acetate succinate-M and about 1% sodium lauryl sulfate.

50. The pharmaceutical composition of claim 34 or 35, wherein the first spray-dried dispersion comprises about 60% wt/wt VX-950, about 39% hypromellose acetate succinate-M and about 1% sodium lauryl sulfate.

51. The pharmaceutical composition of claim 34 or 36, wherein the second spray-dried dispersion comprises about 49.5% wt/wt VX-950, about 49.5% hypromellose acetate succinate-H and about 1% sodium lauryl sulfate.

52. The pharmaceutical composition of any one of claims 18-51, further comprising one or more of a diluent, a disintegrant, a flow agent and a lubricant.

53. The pharmaceutical composition of claim 52, wherein the pharmaceutical composition comprises a diluent.

54. The pharmaceutical composition of claim 53, wherein the diluent is one or more of microcrystalline cellulose and anhydrous dibasic calcium phosphate.

55. The pharmaceutical composition of claim 52, wherein the pharmaceutical composition comprises a disintegrant.

56. The pharmaceutical composition of claim 55, wherein the disintegrant is one or more of microcrystalline cellulose and croscarmellose sodium.

57. The pharmaceutical composition of claim 52, wherein the pharmaceutical composition comprises a flow agent.

58. The pharmaceutical composition of claim 57, wherein the flow agent is colloidal silicon dioxide.

59. The pharmaceutical composition of claim 52, wherein the pharmaceutical composition comprises a lubricant.

60. The pharmaceutical composition of claim 59, wherein the lubricant is sodium stearyl fumarate.

61. A process for preparing a solid dispersion of VX-950, the process comprising:
a) forming a mixture comprising VX-950, a solvent and a polymer selected from the group consisting of hypromellose acetate succinate-M, hypromellose acetate succinate-L and hypromellose acetate succinate-H; and
b) spray-drying the mixture to form a solid dispersion comprising VX-950.

62. The process of claim 61, wherein the solid dispersion comprises from about 40% to about 80% wt/wt VX-950.

63. The process of claim 62, wherein the solid dispersion comprises about 60% wt/wt VX-950.

64. The process of claim 62, wherein the solid dispersion comprises about 70% wt/wt VX-950.

65. The process of any one of claims 61-64, wherein the solid dispersion comprises from about 20% to about 50% wt/wt hypromellose acetate succinate-M.

66. The process of claim 65, wherein the solid dispersion comprises about 29% wt/wt hypromellose acetate succinate-M.

67. The process of claim 65, wherein the solid dispersion comprises about 39% wt/wt hypromellose acetate succinate-M.

68. The process of any one of claims 61-64, wherein the solid dispersion comprises from about 20% to about 50% wt/wt hypromellose acetate succinate-L.

69. The process of claim 68, wherein the solid dispersion comprises about 29% wt/wt hypromellose acetate succinate-L.

70. The process of claim 68, wherein the solid dispersion comprises about 39% wt/wt hypromellose acetate succinate-L.

71. The process of any one of claims 61-64, wherein the solid dispersion comprises from about 20% to about 60% wt/wt hypromellose acetate succinate-H.

72. The process of claim 65, wherein the solid dispersion comprises about 49.5% wt/wt hypromellose acetate succinate-H.

73. The process of any one of claims 61-72, wherein the solid dispersion comprises about 1% wt/wt sodium lauryl sulfate.

74. The process of any one of claims 61-73, wherein the solvent is one or more of methylene chloride, acetone and water.

75. A process for preparing a pharmaceutical composition comprising VX-950, the process comprising:
a) forming a first mixture comprising VX-950, a solvent and a polymer selected from the group consisting of hypromellose acetate succinate-M, hypromellose acetate succinate-L and hypromellose acetate succinate-H;
b) spray-drying the mixture to form a first solid dispersion comprising VX-950;
c) forming a second mixture comprising VX-950, a solvent and a polymer selected from the group consisting of hypromellose acetate succinate-M, hypromellose acetate succinate-L and hypromellose acetate succinate-H;
d) spray-drying the mixture to form a second solid dispersion comprising VX-950; and
e) combining the first and second solid dispersions to form a pharmaceutical composition comprising VX-950.

76. The process of claim 75, wherein the first or second solid dispersion comprises from about 40% to about 80% wt/wt VX-950.

77. The process of claim 76, wherein the first or second solid dispersion comprises about 60% wt/wt VX-950.

78. The process of claim 76, wherein the first or second solid dispersion comprises about 70% wt/wt VX-950.

79. The process of any one of claims 75-78, wherein the first or second solid dispersion comprises from about 20% to about 50% wt/wt hypromellose acetate succinate-M.

80. The process of claim 79, wherein the first or second solid dispersion comprises about 29% wt/wt hypromellose acetate succinate-M.

81. The process of claim 79, wherein the first or second solid dispersion comprises about 39% wt/wt hypromellose acetate succinate-M.

82. The process of any one of claims 75-78, wherein the first or second solid dispersion comprises from about 20% to about 50% wt/wt hypromellose acetate succinate-L.

83. The process of claim 82, wherein the first or second solid dispersion comprises about 29% wt/wt hypromellose acetate succinate-L.

84. The process of claim 82, wherein the first or second solid dispersion comprises about 39% wt/wt hypromellose acetate succinate-L.

85. The process of any one of claims 75-78, where in the first or second solid dispersion comprises from about 20% to about 60% wt/wt hypromellose acetate succinate-H.

86. The process of claim 85, wherein the first or second solid dispersion comprises about 49.5% wt/wt hypromellose acetate succinate-H.

87. The process of any one of claims 75-86, wherein the first or second solid dispersion comprises about 1% wt/wt sodium lauryl sulfate.

88. A method of treating a patient infected with the hepatitis C virus, the method comprising administering the pharmaceutical composition of any one of claims 18-60.

89. The method of claim 88, further comprising administering one or more additional antiviral agents.

90. The method of claim 88, further comprising administering pegylated interferon.

91. The method of claim 88, further comprising administering ribavirin.

92. The method of claim 88, further comprising administering VX-222.

93. The method of any one of claims 88-92, wherein the pharmaceutical composition comprises about 250 mg to about 2250 mg VX-950 per administration.

94. The method of any one of claims 88-93, wherein the pharmaceutical formulation is administered:
a) in an amount of 250 mg VX-950;
b) in an amount of 300 mg VX-950;
c) in an amount of 400 mg VX-950;
d) in an amount of 450 mg VX-950;
e) in an amount of 500 mg VX-950;
f) in an amount of 600 mg VX-950;
g) in an amount of 650 mg VX-950;
h) in an amount of 750 mg VX-950;
i) in an amount of 850 mg VX-950;
j) in an amount of 1000 mg VX-950;
k) in an amount of 1125 mg VX-950;
l) in an amount of 1250 mg VX-950; or
m) in an amount of 2250 mg VX-950.

95. The method of claim 88-94, wherein the pharmaceutical formulation is administered once per day, twice per day or three times per day.

96. The spray-dried dispersion of any one of claims 1-17, wherein the spray-dried dispersion provides an average plasma concentration (C_{avg}) of VX-950 of at least about 750 ng/mL after administration to a human.

97. The spray-dried dispersion of claim 96, wherein the average plasma concentration (C_{avg}) of VX-950 is about 750 ng/mL to about 1250 ng/mL after administration to a human.

98. The pharmaceutical composition of any one of claims 18-60, wherein the pharmaceutical composition provides an average plasma concentration (C_{avg}) of VX-950 of at least about 750 ng/mL after administration to a human.

99. The pharmaceutical composition of claim 98, wherein the average plasma concentration (C_{avg}) of VX-950 is about 750 ng/mL to about 1250 ng/mL after administration to a human.

100. The spray-dried dispersion of any one of claims 1-17, wherein the spray-dried dispersion provides at least about a 2 log₁₀ decrease of hepatitis C virus RNA in the plasma when administered to a human.

101. The spray-dried dispersion of claim 100, wherein the spray-dried dispersion provides at least about a 4 log₁₀ decrease of hepatitis C virus RNA in the plasma when administered to a human.

102. The pharmaceutical composition of any one of claims 18-60, wherein the pharmaceutical composition provides at least about a 2 log₁₀ decrease of hepatitis C virus RNA in the plasma when administered to a human.

103. The pharmaceutical composition of claim 102, wherein the pharmaceutical composition provides at least about a 4 log₁₀ decrease of hepatitis C virus RNA in the plasma when administered to a human.

104. The pharmaceutical composition of any one of claims 18-60, wherein the pharmaceutical composition is substantially bioequivalent to Incivek™.

105. The pharmaceutical composition of any one of claims 18-60, wherein the 90% confidence interval of the relative mean Cₘₐₓ of the pharmaceutical composition to Incivek™ is substantially within 80% to 125%.

106. The pharmaceutical composition of any one of claims 18-60, wherein the 90% confidence interval of the relative mean AUC₍₀₋ₜ₎ of the pharmaceutical composition to Incivek™ is substantially within 80% to 125%.

107. The pharmaceutical composition of any one of claims 18-60, wherein the 90% confidence interval of the relative mean AUC_{(0-∞)} of the pharmaceutical composition to Incivek™ is substantially within 80% to 125%.

108. The spray-dried dispersion of claim 17, wherein the plurality of polymers decreases the amount of crystallization or rate of crystallization of the VX-950 by at least about 10% as compared to a spray-dried dispersion without being in the presence of the plurality of polymers.

109. The pharmaceutical composition of any one of claims 34-36, wherein the plurality of polymers decreases the amount of crystallization or rate of crystallization of the VX-950 by at least about 10% as compared to a pharmaceutical composition without the presence of the plurality of polymers.

110. The pharmaceutical composition of claim 18, wherein the presence of hypromellose acetate succinate-M decreases the amount of crystallization or rate of crystallization of the VX-950 by at least about 10% as compared to a pharmaceutical composition without the presence of hypromellose acetate succinate-M.

111. The pharmaceutical composition of claim 18, wherein the presence of hypromellose acetate succinate-L decreases the amount of crystallization or rate of crystallization of the VX-950 by at least about 10% as compared to a pharmaceutical composition without the presence of hypromellose acetate succinate-L.

112. The pharmaceutical composition of claim 18, wherein the presence of hypromellose acetate succinate-H decreases the amount of crystallization or rate of crystallization of the VX-950 by at least about 10% as compared to a pharmaceutical composition without the presence of hypromellose acetate succinate-H.

113. The pharmaceutical composition of any one of claims 18-60, wherein the pharmaceutical composition further comprises an additional active pharmaceutical ingredient.

114. The pharmaceutical composition of claim 113, wherein the additional active pharmaceutical ingredient is VX-222.
